(19) United States
(12) Patent Application Publication
(10) Pub. No.: US 2025/0331820 A1
(43) Pub. Date: Oct. 30, 2025

(54) AUTOMATIC ARRIVAL TIME DETECTION ALGORITHM FOR ULTRASOUND CONTRAST EXAMINATION

(71) Applicant: GE Precision Healthcare LLC, Waukesha, WI (US)

(72) Inventors: Hiroshi Hashimoto, Hino (JP); Takuma Oguri, Hino (JP); Naohisa Kamiyama, Hino (JP)

(21) Appl. No.: 19/190,287

(22) Filed: Apr. 25, 2025

(30) Foreign Application Priority Data
Apr. 25, 2024 (JP) 2024-071811

(51) Int. Cl. A61B 8/00

(52) U.S. Cl. CPC A61B 8/461; A61B 8/469; A61B 8/481

(57) ABSTRACT

An ultrasound diagnostic device configured to compare, with a threshold value, a pixel value of each pixel included in an ultrasound image; determine the total number of pixels obtained for the ultrasound image of the current time point as a peak value; set a time window, and calculating a change amount at the current time point in the peak value; calculate an index value of a current time point representative of the change amount at the current time point with respect to the peak value of the current time point; determine whether the current time point is an initial arrival time of a contrast agent; and repeatedly execute the process of counting the total number of pixels.

### CROSS-REFERENCE TO RELATED APPLICATION

This application claim priority to Japanese Patent Application No. 2024-071811, which was file on Apr. 25, 2024 at the Japanese Patent Office. The entire contents of the above-listed application are incorporated by reference herein in their entirety.

### TECHNICAL FIELD

The present invention relates to an ultrasound diagnostic device that executes contrast imaging, and to a storing medium containing an instruction to be executed by the ultrasound diagnostic device.

### BACKGROUND

In an ultrasound contrast examination, a contrast agent is administered to a subject and then the examination is performed. There is a certain time difference between the time required for the contrast agent to reach a tumor and a normal site, and this time difference is useful information for making a differential diagnosis. For example, in a normal liver, portal blood flow is dominant, whereas in a typical hepatocellular carcinoma (HCC), arterial blood is dominant. Therefore, when a contrast agent is administered, the contrast agent reaches the HCC first, and the liver parenchyma is stained after the contrast agent reaches the HCC. As a method for quantitatively presenting the time difference, a method using a TIC (Time Intensity Curve) and a parametric image is known. A parametric image is often used as a function for displaying the arrival time of a contrast agent in color.

### SUMMARY

A first aspect of the present invention is an ultrasound diagnostic device including one or a plurality of processors that executes the following: comparing, with a threshold value, a pixel value of each pixel included in an ultrasound image acquired at a current time point and counting the total number of pixels having a pixel value greater than the threshold value; determining the total number of pixels obtained for the ultrasound image of the current time point as a peak value if the total number of pixels obtained for the ultrasound image of the current time point is greater than the maximum value of the total number of pixels obtained in the past, and performing a peak hold for holding the peak value at a time point immediately preceding the current time point as the peak value at the current time point if the total number of pixels obtained for the ultrasound image of the current time point is at the maximum value or less of the total number of pixels obtained in past; setting a time window including the peak value of the current time point and a past peak value, and calculating a change amount at the current time point in the peak value on the basis of a plurality of peak values included in the time window; calculating an index value of a current time point representative of the change amount at the current time point with respect to the peak value of the current time point; determining whether the current time point is an initial arrival time of a contrast agent on the basis of the index value of the current time point and the maximum value of the index values obtained in the past; and repeatedly executing the process of counting the total number of pixels, the process of executing the peak hold, the process of calculating the change amount, the process of calculating the index value, and the process of determining, and then updating the initial arrival time of the contrast agent every time the current time is deemed to be the initial arrival time of the contrast agent.

A second aspect of the present invention is a non-transitory computer-readable storing medium in which an instruction is stored, wherein the instruction, when executed by one or a plurality of processors, causes the one or plurality of processors to execute the following: comparing, with a threshold value, a pixel value of each pixel included in an ultrasound image acquired at a current time point and counting the total number of pixels having a pixel value greater than the threshold value; determining the total number of pixels obtained for the ultrasound image of the current time point as a peak value if the total number of pixels obtained for the ultrasound image of the current time point is greater than the maximum value of the total number of pixels obtained in the past, and performing a peak hold for holding the peak value at a time point immediately preceding the current time point as the peak value at the current time point if the total number of pixels obtained for the ultrasound image of the current time point is at the maximum value or less of the total number of pixels obtained in past; setting a time window including the peak value of the current time point and a past peak value, and calculating a change amount at the current time point in the peak value on the basis of a plurality of peak values included in the time window; calculating an index value of a current time point representative of the change amount at the current time point with respect to the peak value of the current time point; determining whether the current time point is an initial arrival time of a contrast agent on the basis of the index value of the current time point and a maximum value of the index values obtained in the past; and repeatedly executing the step of counting the total number of pixels, the step of executing the peak hold, the step of calculating the change amount, the step of calculating the index value, and the step of determining, and then updating the initial arrival time of the contrast agent every time the current time is deemed to be the initial arrival time of the contrast agent.

### BRIEF DESCRIPTION OF THE DRAWINGS

FIG. 1 is a block diagram of the ultrasound diagnostic device 1;

FIG. 2 is a diagram depicting a curve G1 representing the total number of pixels stained by a contrast agent;

FIG. 3 is a diagram depicting a peak hold curve G2 obtained by executing a peak hold on the total number of pixels of the curve G1;

FIG. 4 is a diagram depicting a change amount curve G3 representing a change amount of a peak value;

FIG. 5 is an explanatory diagram of a method for calculating a change amount c;

FIG. 6 is a diagram depicting a curve G4 representing an index value;

FIG. 7 is a flowchart of a process of detecting an initial arrival time of a contrast agent;

FIG. 8 is an explanatory diagram of steps ST41 to ST47 when executing the process of detecting the arrival time of the contrast agent on the basis of an ultrasound image U₁ at a current time point t₁;

FIG. 9 is an explanatory diagram of step ST41;

FIG. 10 is an explanatory diagram of steps ST41 to ST47 when executing the process of detecting the arrival time of the contrast agent on the basis of an ultrasound image U₂ at a current time point t₂;

FIG. 11 is an explanatory diagram of a method for creating a parametric image E₂ at the time point t₂;

FIG. 12 is an explanatory diagram of step ST40 at a current time point t₃;

FIG. 13 is an explanatory diagram of a method for creating a parametric image E₃ at the time point t₃;

FIG. 14 is an explanatory diagram of step ST40 at a time point t₄;

FIG. 15 is an explanatory diagram of step ST48;

FIG. 16 is an explanatory diagram of step ST40 at a time point t₅;

FIG. 17 is an explanatory diagram of a method for creating a parametric image Es at the time point t₅;

FIG. 18 is an explanatory diagram of step ST40 at a time point t₆;

FIG. 19 is an explanatory diagram of a method for creating a parametric image E₆ at the time point t₆;

FIG. 20 is an explanatory diagram of step ST40 at a time point t₇;

FIG. 21 is an explanatory diagram of step ST48;

FIG. 22 is an explanatory diagram of step ST40 at a time point t₈;

FIG. 23 is an explanatory diagram of a method for creating a parametric image E₈ at the time point t₈;

FIG. 24 is an explanatory diagram of step ST40 at a time point t₉;

FIG. 25 is an explanatory diagram of a method for creating a parametric image E₉ at the time point t₉;

FIG. 26 is an explanatory diagram of step ST40 at a time point t₁₀;

FIG. 27 is an explanatory diagram of a method for creating a parametric image E₁₀ at the time point t₁₀;

FIG. 28 is an explanatory diagram of step ST40 at a time point t₁₁;

FIG. 29 is an explanatory diagram of a method for creating a parametric image E₁₁ at the time point t₁₁; and

FIG. 30 is a diagram depicting data obtained between time points t₁ and t₂₅.

### DETAILED DESCRIPTION

When performing a contrast examination, an examiner scans a patient to acquire a series of ultrasound images in chronological order. After completing the contrast examination, the examiner analyzes the acquired ultrasound images and determines an initial arrival time, which represents the time when a contrast agent first flows into an examination site. A parametric image is then created on the basis of the initial arrival time.

However, when determining the initial arrival time of the contrast agent, a user must carefully examine a series of ultrasound images acquired in the contrast examination, which poses the problem of time-consuming analysis of the ultrasound images.

Therefore, there is a demand for a technology capable of shortening the time required for analyzing an ultrasound image.

In the present invention, an index value is calculated every time an ultrasound image is acquired, and whether the current time is the initial arrival time of a contrast agent is determined on the basis of the index value of the current time point and a maximum value of past index values. Therefore, the initial arrival time of the contrast agent can be automatically detected in real-time while a contrast examination is being performed on the subject. This eliminates the need for a user to analyze acquired ultrasound images to determine the initial arrival time of the contrast agent, thereby reducing the workload on the user during a contrast examination and shortening the time required to analyze ultrasound images.

Embodiments for carrying out the invention will be described below, but the present invention is not limited to the following embodiments.

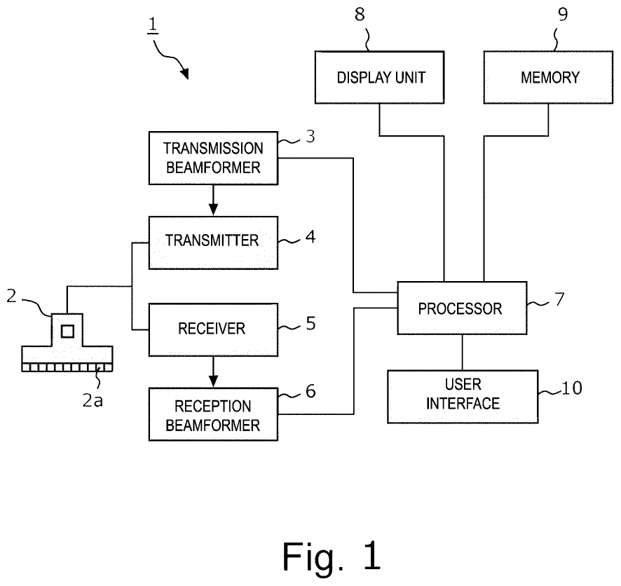

FIG. 1 is a block diagram of an ultrasound diagnostic device 1.

The ultrasound diagnostic device 1 has an ultrasonic probe 2, a transmission beamformer 3, a transmitter 4, a receiver 5, a reception beamformer 6, a processor 7, a display unit 8, a memory 9, and a user interface 10.

The ultrasonic probe 2 has a plurality of vibrating elements 2a arranged in an array. The transmission beamformer 3 and the transmitter 4 drive the plurality of vibrating elements 2a, which are arrayed within the ultrasonic probe 2, and ultrasonic waves are transmitted from the vibrating elements 2a. The ultrasonic waves transmitted from the vibrating element 2a are reflected inside the subject, and a reflection echo is received by the vibrating element 2a. The vibrating elements 2a convert the received echo to an electrical signal and output this electrical signal as an echo signal to the receiver 5. The receiver 5 executes a prescribed process on the echo signal and outputs the echo signal to the reception beamformer 6. The reception beamformer 6 executes reception beamforming on the signal received through the receiver 5 and outputs echo data.

The reception beamformer 6 may be a hardware beamformer or a software beamformer. If the reception beamformer 6 is a software beamformer, the reception beamformer 6 may include one or a plurality of processors, including one or a plurality of: i) a graphics processing unit (GPU); ii) a microprocessor; iii) a central processing unit (CPU); iv) a digital signal processor (DSP); or v) another type of processor capable of executing logical operations. A processor configuring the reception beamformer 6 may be configured by a processor different from the processor 7 or may be configured by the processor 7.

The ultrasonic probe 2 may include an electrical circuit for performing all or a portion of transmission beamforming and/or reception beamforming. For example, all or a portion of the transmission beamformer 3, the transmitter 4, the receiver 5, and the reception beamformer 6 may be provided in the ultrasonic probe 2.

The processor 7 controls the transmission beamformer 3, the transmitter 4, the receiver 5, and the reception beamformer 6. Furthermore, the processor 7 is in electronic communication with the ultrasonic probe 2. The processor 7 controls which of the vibrating elements 2a is active and the shape of ultrasonic beams transmitted from the ultrasonic probe 2. The processor 7 is in electronic communication with the display unit 8. The processor 7 can process echo data to generate an ultrasound image. The term “electronic communication” may be defined to include both wired and wireless communications. The processor 7 may include a central processing unit (CPU) according to one embodiment. According to another embodiment, the processor 7 may include one or more processor, another electronic component that may execute a processing function such as a digital signal processor, a field programmable gate array (FPGA), a graphics processing unit (GPU), another type of processor, and the like. According to another embodiment, the processor 7 may include a plurality of electronic components capable of executing a processing function. For example, the processor 7 may include two or more electronic components selected from a list of electronic components including a central processing unit, a digital signal processor, a field programmable gate array, and a graphics processing unit.

The processor 7 may also include a complex demodulator (not depicted) that demodulates RF data. In another embodiment, demodulation may be executed in an earlier stage in the processing chain.

Furthermore, the processor 7 may generate various ultrasound images (e.g., a B-mode image, color Doppler image, M-mode image, color M-mode image, spectral Doppler image, elastography image, TVI image, strain image, strain rate image, and the like) on the basis of data obtained by processing via the reception beamformer 6. In addition, one or a plurality of modules can generate these ultrasound images.

An image beam and/or an image frame may be saved, and timing information may be recorded indicating when the data is retrieved to the memory. The module may include, for example, a scan conversion module that executes a scan conversion operation to convert an image frame from a coordinate beam space to display space coordinates. A video processor module may also be provided for reading an image frame from the memory while a procedure is being implemented on the subject and displaying the image frame in real-time. The video processor module may save the image frame in an image memory, and the ultrasonic images may be read from the image memory and displayed on the display unit 8.

In the present Specification, the term “image” can broadly indicate both a visual image and data representing a visual image. Furthermore, the term “data” can include raw data, which is ultrasound data before a scan conversion operation, and image data, which is data after the scan conversion operation.

Note that the processing tasks described above handled by the processor 7 may be executed by a plurality of processors.

Furthermore, when the reception beamformer 6 is a software beamformer, a process executed by the beamformer may be executed by a single processor or may be executed by the plurality of processors.

Examples of the display unit 8 include LED (Light-Emitting Diode) display units, LCDs (Liquid Crystal Display), and organic EL (Electro-Luminescence) display units. The display unit 8 displays an ultrasound image.

The memory 9 is any known data storing medium. In one example, the ultrasound image display system includes a non-transitory storing medium and a transitory storing medium as memories. In addition, the ultrasound image display system may also include a plurality of memories. The non-transitory storing medium is, for example, a non-volatile storing medium such as a Hard Disk Drive (HDD), a Read-Only Memory (ROM), or the like. The non-transitory storing medium may include a portable storing medium such as a CD (Compact Disk), a DVD (Digital Versatile Disk), or the like. A program executed by the processor 7 is stored in the non-transitory storing medium. The transitory storing medium is a volatile storing medium such as a Random-Access Memory (RAM) or the like.

The memory 9 stores one or a plurality of instructions that can be executed by the processor 7. The one or plurality of instructions cause the processor 7 to execute various types of operations.

Note that the processor 7 may also be configured so as to be able to connect to an external storing device by a wired connection or a wireless connection. In this case, the instruction causing execution by the processor 7 can be distributed to both the memory 9 and the external storing device for storage.

The user interface 10 can receive input from a user (e.g., an operator). For example, the user interface 10 receives instruction or information input by the user. The user interface 10 is configured to include a keyboard (keyboard), a hard key (hard key), a trackball (trackball), a rotary control (rotary control), a soft key, and the like. The user interface 10 may include a touch screen that displays a soft key or the like.

The ultrasound diagnostic device 1 is configured as described above.

Before specifically describing an embodiment of the ultrasound diagnostic device 1, a basic principle of a method for detecting an arrival time of a contrast agent in the present embodiment will be described.

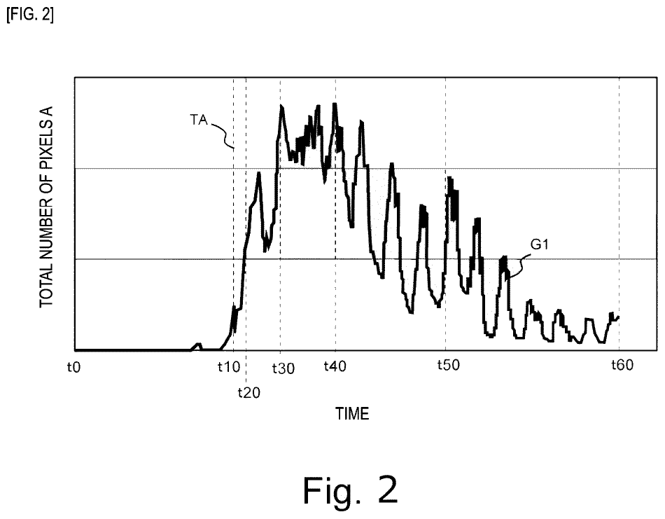

FIGS. 2 to 6 are explanatory diagrams of the basic principle of the method for detecting an arrival time of a contrast agent.

FIG. 2 depicts a curve G1. The horizontal axis represents time, and the vertical axis represents the total number of pixels stained by the contrast agent among a plurality of pixels included in an ultrasound image. Therefore, the curve G1 schematically represents how the total number of pixels stained by the contrast agent changes over time.

A time point to represents a scanning start time point. The contrast agent may be administered immediately before the scanning start time point to, simultaneously with the scanning start time point to, or immediately after the scanning start time point to. Immediately after the start of scanning, the contrast agent has not yet flowed into an examination site, and therefore, a total number a of pixels stained by the contrast agent is zero. However, as time passes and the contrast agent starts to flow into the examination site, the total number of pixels stained by the contrast agent rapidly increases, causing the curve to steeply rise. A time point to at which the curve begins to steeply rise represents an initial arrival time TA of the contrast agent. After the initial arrival time TA of the contrast agent has passed, the total number a of pixels stained by the contrast agent increases, and the curve G1 reaches a peak at a time point t₄₀. Furthermore, the contrast agent gradually flows out of the examination site, and therefore, the total number of pixels stained by the contrast agent decreases over time.

As described above, if the contrast agent starts to flow into the examination site, the total number of pixels stained by the contrast agent rapidly increases. Therefore, the time point t₁₀ at which the change amount in the curve rapidly increases can be identified, and that time point t₁₀ can be regarded as the initial arrival time TA of the contrast agent.

However, even after the initial arrival time TA of the contrast agent has passed, the total number a of pixels increases or decreases. Therefore, even after the initial arrival time TA has passed, a time point appears where the total number a of pixels stained by the contrast agent increases rapidly. For example, referring to time point t₄₀ to time point t₆₀, the time from time point t₄₀ and thereafter represents a time phase in which the contrast agent flows out from the examination site, and therefore, the total number a of pixels decreases over time. However, the total number a of pixels does not monotonically decrease but repeatedly increases and decreases, and therefore, a time point (e.g., time point t₅₀) appears between the time point t₄₀ and time point too at which the total number of pixels stained by the contrast agent rapidly increases.

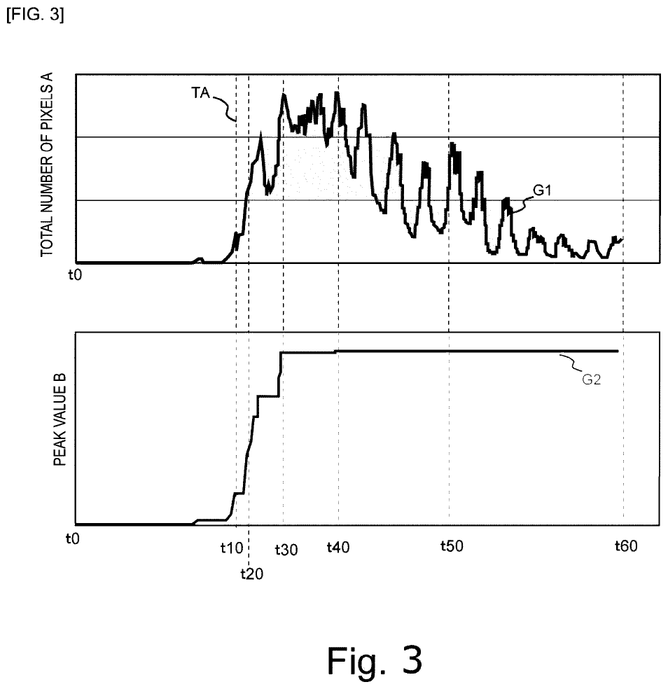

Therefore, in order to determine the initial arrival time TA of the contrast agent, a distinction must be made between the rapid increase in the total number of pixels that occurs at the initial arrival time TA of the contrast agent and the rapid increase in the total number of pixels that occurs between the time point t₄₀ and time point t₆₀. Therefore, in order to make this distinction, the inventor of the present application conceived of executing a peak hold with respect to the total number a of pixels of the curve G1 (see FIG. 3).

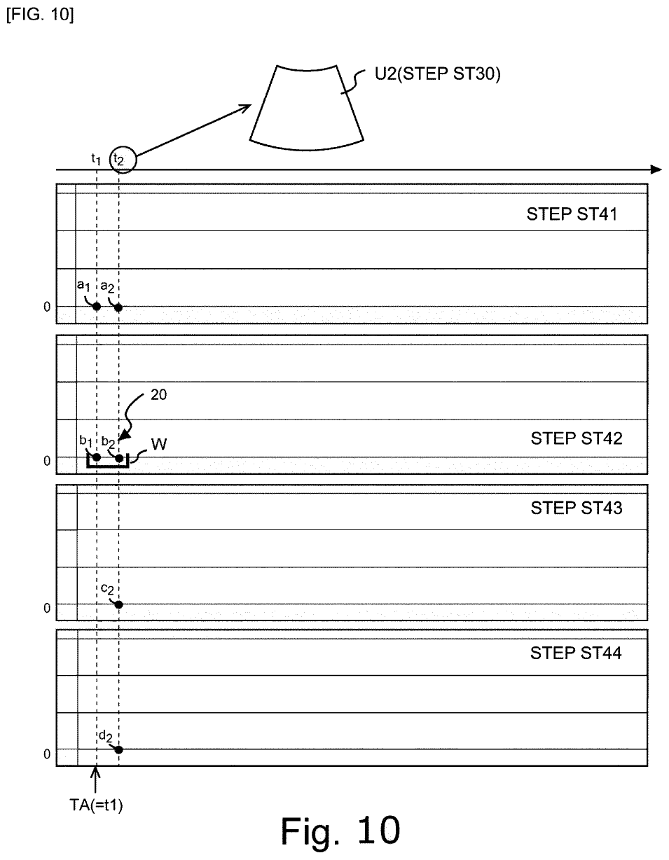

FIG. 3 is a diagram depicting a peak hold curve G2 obtained by executing a peak hold with respect to the total number of pixels of the curve G1 (note that details of the peak hold are described in FIG. 10 and the like, which will be described later).

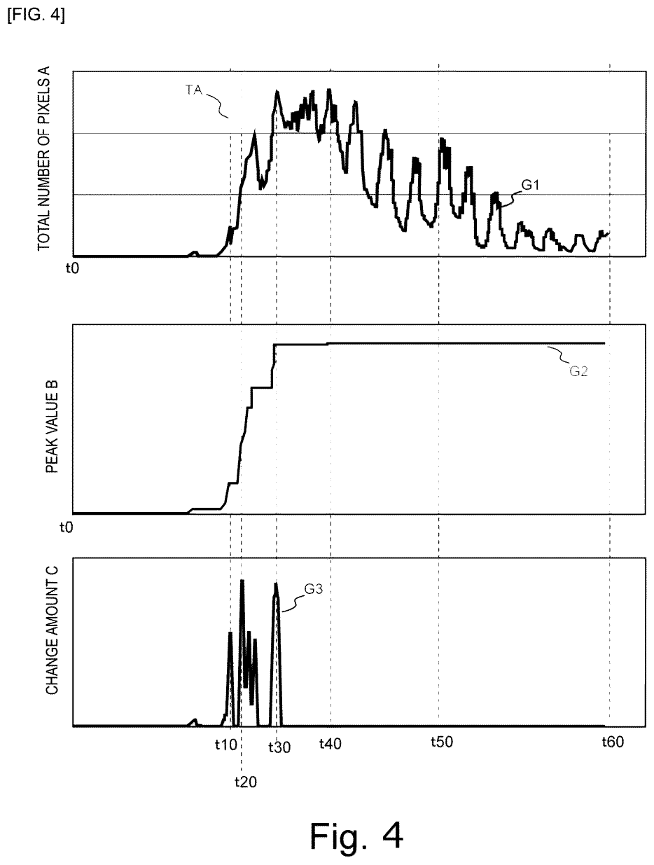

The peak hold curve G2 steeply rises at the initial arrival time TA of the contrast agent (time point t10), but after the total number a of pixels reaches a maximum value at time point t₄₀, the same value is maintained regardless of whether the total number a of pixels increases or decreases. Therefore, in the peak hold curve G2, a clear difference appears between the change amount in the peak value at the time point t₁₀ and in the vicinity thereof, and the change amount in the peak value from time points t₄₀ to t₆₀. In order to make this difference easier to understand, FIG. 4 depicts a change amount curve G3 representing the change amount in the peak value.

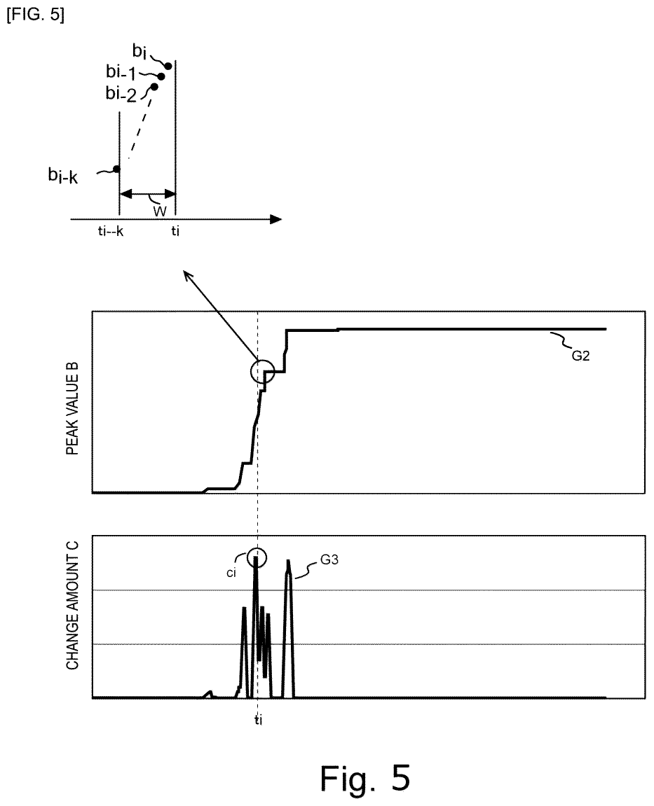

A change amount c of the change amount curve G3 can be calculated on the basis of the peak hold curve G2. FIG. 5 is an explanatory diagram of a method for calculating the change amount c. For example, a case of calculating the change amount cᵢ at an arbitrary time point ti of the change amount curve G3 is considered. When calculating the change amount cᵢ, a time window W having a prescribed time width is set in the peak hold curve G2. The time window W includes (k+1) number of peak values bᵢ₋ₖ to bᵢ included in time points tᵢ₋ₖ to tᵢ. The change amount cᵢ at the time point tᵢ can be calculated by the change amount (slope) of the (k+1) number of peak values bᵢ₋ₖ to bᵢ.

Returning to FIG. 4, the description is continued.

Therefore, referring to the change amount curve G3, the change amount at time point t₁₀ is a large positive value, but after the time point t₄₀, the change amount is zero. Thus, by analyzing the value of the change amount curve G3, the initial arrival time TA (t₁₀) can be distinguished from the time points t₄₀ to t₆₀.

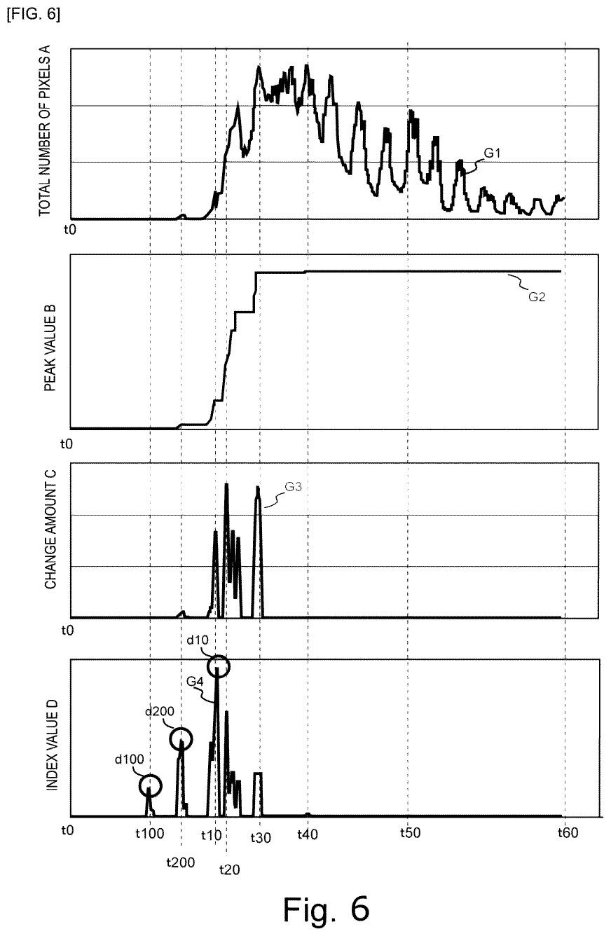

However, in the change amount curve G3, the change amount is large not only at the initial arrival time TA (t₁₀) of the contrast agent, but also at time point t₂₀ and time point t₃₀. Therefore, in order to specify the initial arrival time TA (t₁₀) of the contrast agent, it is necessary to distinguish between the initial arrival time TA (t₁₀) of the contrast agent and time points t₂₀ and t₃₀. Therefore, in order to make this distinction, the inventors of the present application conceived of calculating an index value that reflects both the peak value b and the change amount c. FIG. 6 depicts a curve G4 representing an index value. The index value can be calculated using the following equation (1).

$${d}_{i} = {c}_{i} / {b}_{i} \qquad ( 1 )$$

Herein, dᵢ represents the index value at the time point tᵢ, cᵢ represents the change amount at the time point tᵢ, and bᵢ represents the peak value at the time point tᵢ. Therefore, the larger the peak value bᵢ, the smaller the index value dᵢ. The initial arrival time TA (t₁₀) of the contrast agent is a time phase when the contrast agent starts to flow into the examination site, and therefore, the peak value bᵢ does not become a very large value at the initial arrival time TA (t₁₀) of the contrast agent. Therefore, at the initial arrival time TA (t₁₀) of the contrast agent, the index value dᵢ becomes a large value. On the other hand, at the time point t₂₀ and time point t₃₀, the region stained by the contrast agent expands, such that the peak value bᵢ becomes large. Therefore, the index value dᵢ reaches a maximum value at the initial arrival time TA (t₁₀) of the contrast agent, and starts to decrease at and after the initial arrival time TA (t₁₀) of the contrast agent. Therefore, by calculating the index value dᵢ, the initial arrival time TA (t₁₀) of the contrast agent can be distinguished from time point t₂₀ and time point t₃₀.

Note that even when the contrast agent has not yet reached tissue, there are pixels with high brightness due to the nature of the tissue. Even if the pixels having this high brightness are not stained with the contrast agent, the pixels behave in the same manner as if stained with the contrast agent. Therefore, referring to the index value curve G4, at time points t₁₀₀ and t₂₀₀ before the arrival of the contrast agent (before time point t₁₀), the index values d₁₀₀ and d₂₀₀ indicate values greater than zero. However, the index values d₁₀₀ and d₂₀₀ at the time points t₁₀₀ and t₂₀₀ when the contrast agent has not yet reached are sufficiently smaller than the index value d₁₀ at the initial arrival time TA (t₁₀) of the contrast agent. Therefore, the index value d₁₀ at the initial arrival time TA (t₁₀) of the contrast agent can be distinguished from the index values d₁₀₀ and d₂₀₀ at a time point when no contrast agent has not arrived.

Therefore, by calculating the index value dᵢ as described above, the initial arrival time of the contrast agent can be identified. A method for detecting the initial arrival time of the contrast agent using the aforementioned index value dᵢ will be described below.

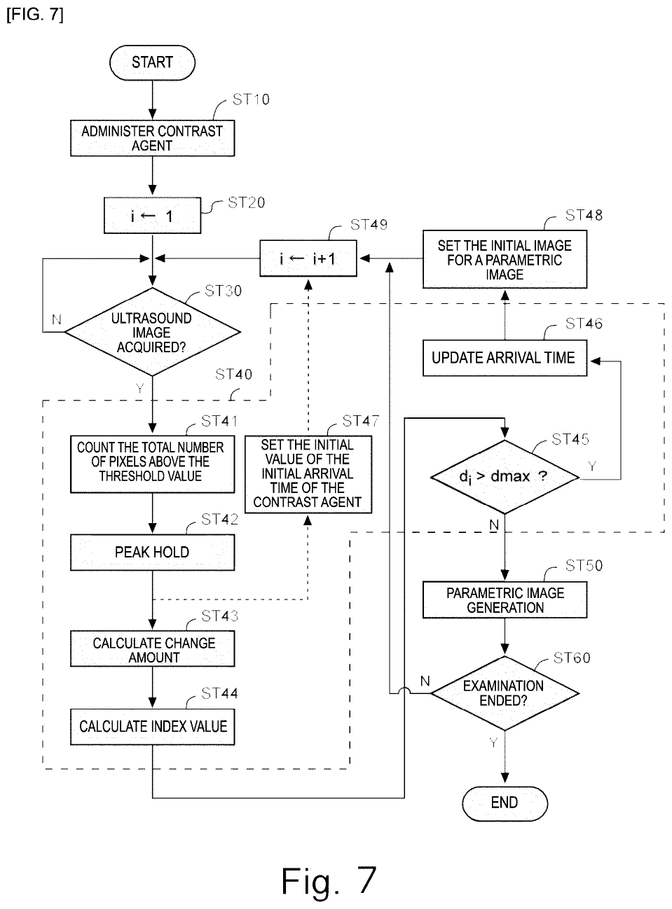

FIG. 7 is a flowchart of a process of detecting the initial arrival time of the contrast agent.

Note that in the following description, priority is given to making the feature portion of the embodiment easier to understand, and the number of data and values of each piece of data differ from the number of data and data values in an actual contrast examination.

In step ST10, a contrast agent is administered to a subject.

In step ST20, i is set to initial value (i=1). i represents the indexes of a time point tᵢ, ultrasound image Uᵢ, total number aᵢ of pixels, peak value bᵢ, change amount cᵢ, and index value dᵢ, which will be described later. After i is set to i=1, the process proceeds to step ST30.

In step ST30, a processor determines whether or not the ultrasound image Uᵢ has been acquired at the current time point tᵢ. Herein, i=1, and therefore, it is determined whether or not the ultrasound image U₁ has been acquired at the current time point t₁. If it is determined that the ultrasound image U₁ has been acquired at the current time point t₁, the process proceeds to step ST40.

In step ST40, a process of detecting the arrival time of the contrast agent is executed on the basis of the ultrasound image U₁ at the current time point t₁.

Note that step ST40 includes steps ST41 to ST47, and thus each step will be described in order.

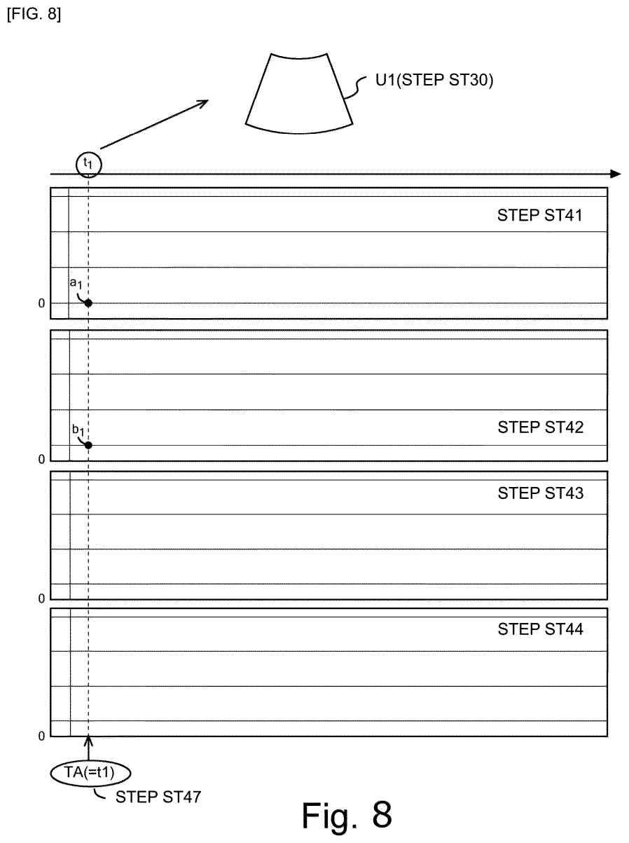

FIG. 8 is an explanatory diagram of steps ST41 to ST47 when executing the process of detecting the arrival time of the contrast agent on the basis of the ultrasound image U₁ at the current time point t₁.

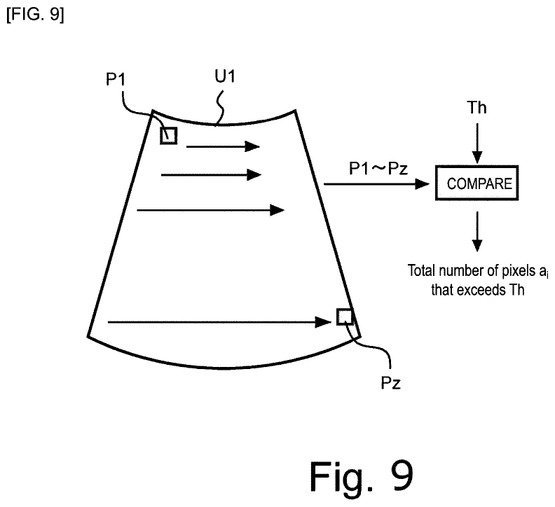

In step ST41, the processor counts the total number a₁ of pixels having pixel values that exceed a threshold value TH among a plurality of pixels included in the ultrasound image U₁ acquired at the current time point t₁ (see FIG. 9).

FIG. 9 is an explanatory diagram of step ST41.

The processor compares the pixel values of each of a plurality of pixels P₁ to P_{z} included in the ultrasound image U₁ with the threshold value Th, and determines that pixels having a pixel value that exceeds the threshold value Th are stained with the contrast agent, while determining that pixels having a pixel value that is at the threshold value Th or less are not stained with the contrast agent. In the present embodiment, the threshold value Th is determined on the basis of the maximum value M of pixel values that can be displayed by each pixel. Specifically, the threshold value This determined on the basis of the following equation.

$$Th = k \cdot M$$

Herein, k is a coefficient having a value in a range 0<k<1.

The value of k can be determined on the basis of the actual pixel value and the like of a pixel when the pixel is stained with the contrast agent. For example, k can be k=0.3. If the maximum value M is M=255 and k=0.3, the threshold value Th can be set to Th=76.5.

Therefore, the processor can determine that the contrast agent has not reached the pixel when the pixel value is smaller than the threshold value Th, and that the contrast agent has reached the pixel when the pixel value exceeds the threshold value Th. Therefore, the processor can determine the total number of pixels where the contrast agent is considered to have reached by counting the total number of pixels having pixel values greater than the threshold value Th among the pixels P₁ to P_{z}. Herein, all pixels in the ultrasound image U₁ have pixel values smaller than the threshold value Th. Therefore, for the ultrasound image U₁, the processor determines the total number a₁ of pixels that exceed the threshold value Th satisfies a₁=0. FIG. 8 depicts the total number a₁ (=0) of pixels at the time point t₁ determined in step ST41. Once the total number a₁ of pixels has been determined, the process proceeds to step ST42.

In step ST42, the processor executes a peak hold on the total number a₁ of pixels at the current time point t₁ to determine the peak value b₁. At the current time point t₁, there is no data on the total number of pixels obtained at a past time, and therefore, the processor sets the total number a₁ of pixels at the current time point t₁ to the peak value b₁ at the current time point t₁. In other words, it is determined that b₁=a₁. Once the peak value b₁ is determined, the process proceeds to step ST43.

Note that at the time point t₁, only one peak value data point (peak value b₁) is acquired. In this case, the process of step ST43 cannot be performed, and therefore, the process does not proceed to step ST43 but proceeds to step ST47.

In step ST47, the processor determines an initial value of the initial arrival time TA of the contrast agent. Herein, the initial value of the initial arrival time TA of the contrast agent is determined to satisfy TA=t₁. After TA=t₁ is determined, the process proceeds to step ST48.

In step ST48, the processor increments i from i=1 to i=2. After i is incremented, the process returns to step ST30.

In step ST30, a processor determines whether or not the ultrasound image Uᵢ has been acquired at the current time point tᵢ. Herein, i has been incremented to i=2, and therefore, it is determined whether or not an ultrasound image U₂ has been acquired at the current time point t₂. If it is determined that the ultrasound image U₂ has been acquired at the current time point t₂, the process proceeds to step ST40.

In step ST40, a process of detecting the arrival time of the contrast agent is executed on the basis of the ultrasound image U₂ at the current time point t₂.

FIG. 10 is an explanatory diagram of steps ST41 to ST47 when executing the process of detecting the arrival time of the contrast agent on the basis of the ultrasound image U₂ at the current time point t₂.

In step ST41, the processor determines the total number a₂ of pixels at the time point t₂. The method for calculating the total number of pixels is the same as the described method with reference to FIG. 9. Herein, a₂=0, similar to time point t₁. Once the total number a₂ of pixels has been calculated, the process proceeds to step ST42.

In step ST42, the processor executes a peak hold on the total number a₂ of pixels at the current time point t₂ to determine a peak value b₂. Specifically, the peak value b₂ is determined as follows.

The processor compares the total number a₂ of pixels obtained for the ultrasound image U₂ at the current time point t₂ with the maximum value aₘₐₓ of the total number of pixels obtained in the past. Furthermore, if the total number a₂ of pixels obtained for the ultrasound image U₂ at the current time point t₂ is greater than the maximum value aₘₐₓ of the total number of pixels obtained in the past (a₂>aₘₐₓ), the processor determines the total number a₂ of pixels obtained for the ultrasound image U₂ at the current time point t₂ as the peak value b₂. On the other hand, if the total number a₂ of pixels obtained for the ultrasound image U₂ at the current time point t₂ is at the maximum value aₘₐₓ or less of the total number of pixels obtained in the past (a₂≤aₘₐₓ), the peak value b₁ at the time point t₁ is retained as the peak value b₂ at the current time point t₂.

Herein, the total number a₂ of pixels at the time point t₂ is the same as the maximum value aₘₐₓ of the total number of pixels obtained in the past (i.e., the same as a₁). Therefore, a₂≤aₘₐₓ, and thus the processor holds the peak value b₁ at time point t₁ as the peak value b₂ at the current time point t₂. Therefore, the peak value b₂ is determined to satisfy b₂=b₁=0. After the peak value b₂ is determined, the process proceeds to step ST43.

In step ST43, a change amount c₂ in the peak value at time point t₂ is calculated. As described with reference to FIG. 5, the change amount is calculated on the basis of the (k+1) number of peak values bᵢ₋ₖ to bᵢ included in a time window W having a prescribed time width. For convenience of description, k is set to 1 in this example. In other words, in the present embodiment, the time window W has a time width that includes two peak values (i.e., the peak value bᵢ at the current time tᵢ and the peak value bᵢ₋₁ at time tᵢ₋₁ immediately preceding the current time point tᵢ).

Therefore, when calculating the change amount c₂ at time point t₂, the processor sets the time window W including the number of data points for two points (i.e., a time window including the peak value b₂ at the current time point t₂ and the peak value b₁ at time point t₁ immediately preceding the current time point t₂), and calculates the change amount c₂ between the two data points b₁ and b₂ included in the time window W. Here, b₂=b₁, so the change amount c₂ satisfies c₂=0. After calculating the change amount c₂, the process proceeds to step ST44.

An index value d₂ at the current time point t₂ is calculated in step ST44. As described with reference to FIG. 6, the index value dᵢ at time point tᵢ is a value calculated by dᵢ=cᵢ/bᵢ. Therefore, the index value d₂ at the current time point t₂ can be calculated by d₂=c₂/b₂. Here, c₂=0, so d₂=0. After calculating the index value d₂, the process proceeds to step ST45.

In step ST45, the processor determines whether or not to update the initial arrival time of the contrast agent on the basis of the index value dᵢ at the current time point tᵢ and the maximum value dₘₐₓ of the past index values. Here, only one index value (index value d₂) has been calculated thus far, so no past index values exist. In this case, the process proceeds to step ST50.

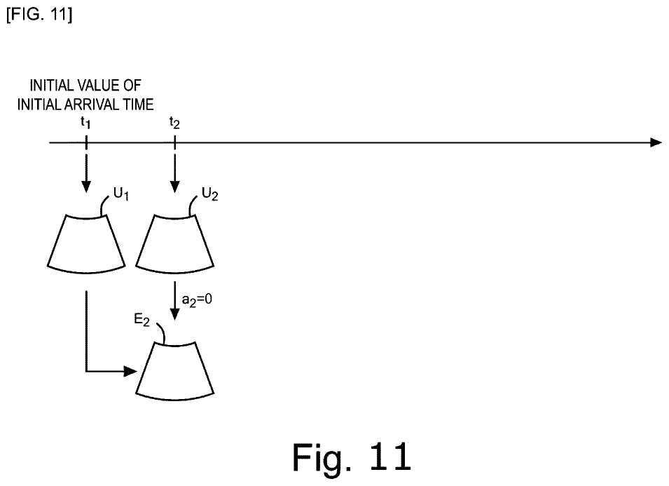

In step ST50, the processor creates a parametric image at time point t₂. A parametric image is an image in which a color corresponding to the arrival time of the contrast agent is assigned to each pixel reached by the contrast agent. FIG. 11 is an explanatory diagram of a method for creating a parametric image E₂ at time point t₂. At time point t₂, the total number a₂ of pixels exceeding the threshold value TH satisfies a₂=0. In this case, no pixels were stained with contrast agent, so the ultrasound image U₁ at time point t₁ is displayed as is as the parametric image E₂ at the current time point t₂. Once the parametric image E₂ is displayed, the process proceeds to step ST60.

In step ST60, whether or not the examination has ended is determined. Herein, the examination has not yet ended, and thus the process proceeds to step ST49.

In step ST49, the processor increments i. Herein, i=2, and thus i is incremented to 3. The process then returns to step ST30.

In step ST30, a processor determines whether or not the ultrasound image Uᵢ has been acquired at the current time point tᵢ. Herein, i has been incremented to i=3, and therefore, it is determined whether or not an ultrasound image U₃ has been acquired at the current time point t₃. If it is determined that the ultrasound image U₃ has been acquired at the current time point t₃, the process proceeds to step ST40.

In step ST40, a process of detecting the arrival time of the contrast agent is executed on the basis of the ultrasound image U₃ at the current time point t₃.

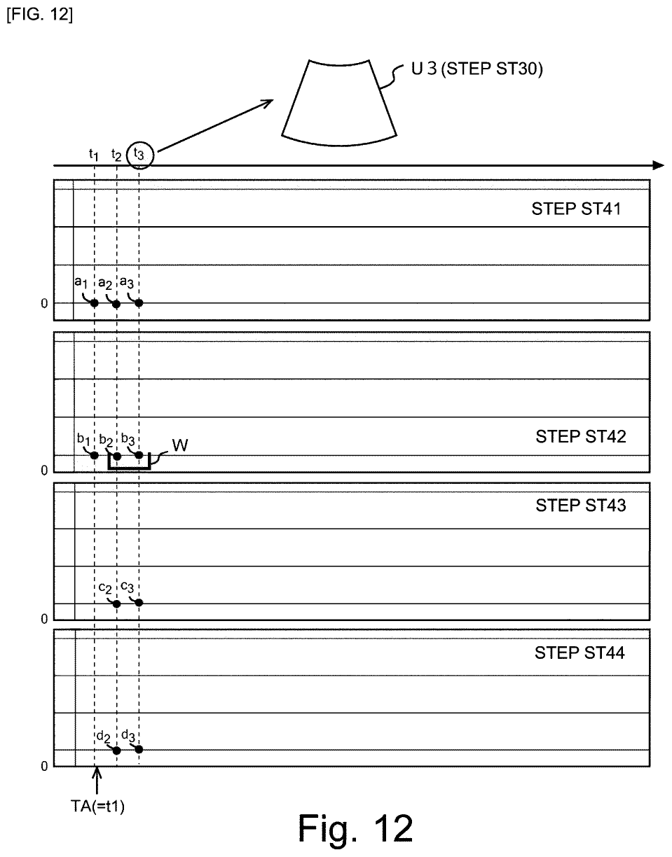

FIG. 12 is an explanatory diagram of step ST40 (steps ST41 to ST47) at the current time point t₃.

In step ST41, the processor determines the total number a₃ of pixels at the time point t₃. The method for calculating the total number of pixels is the same as the described method with reference to FIG. 9. Herein, a₃=0, similar to time point t₂. Once the total number a₃ of pixels has been calculated, the process proceeds to step ST42.

In step ST42, the processor executes a peak hold on the total number a₃ of pixels at the current time point t₃ to determine the peak value b₃. Specifically, the peak value b₃ is determined as follows.

The processor compares the total number a₃ of pixels obtained for the ultrasound image U₃ at the current time point t₃ with the maximum value aₘₐₓ of the total number of pixels obtained in the past. Furthermore, if the total number a₃ of pixels obtained for the ultrasound image U₃ at the current time point t₃ is greater than the maximum value aₘₐₓ of the total number of pixels obtained in the past (a₃>aₘₐₓ), the processor determines the total number a₃ of pixels obtained for the ultrasound image U₃ at the current time point t₃ as the peak value b₃. On the other hand, if the total number a₃ of pixels obtained for the ultrasound image U₃ at the current time point t₃ is at the maximum value aₘₐₓ or less of the total number of pixels obtained in the past (a₃≤aₘₐₓ), the peak value b₂ at the time point t₂ is retained as the peak value b₃ at the current time point t₃.

Herein, the total number a₃ of pixels at the time point t₃ is the same as the maximum value aₘₐₓ of the total number of pixels obtained in the past (i.e., the same as a₁ and a₂). Therefore, a₃≤aₘₐₓ, and thus the processor holds the peak value b₂ at time point t₂ as the peak value b₃ of the total number of pixels at the current time point t₃. Therefore, the peak value b₃ is determined to satisfy b₃=b₂=0. After the peak value b₃ is determined, the process proceeds to step ST43.

In step ST43, a change amount c₃ in the peak value is calculated at time point t₃. Specifically, the processor sets the time window W including two pieces of data (i.e., a time window including the peak value b₃ at the current time point t₃ and the peak value b₂ at the time point t₂ immediately preceding the current time point t₃), and calculates the change amount c₃ between the two pieces of data b₂ and b₃ included in the time window W. Here, b₂=b₃, so the change amount c₃ of the peak value satisfies c₃=0. After calculating the change amount c₃ of the peak value, the process proceeds to step ST44.

In step ST44, an index value d₃ at the current time point t₃ is calculated. The index value d₃ at the current time point t₃ can be calculated by d₃=c₃/b₃. Here, c₃=0, so d₃=0. After calculating the index value d₃, the process proceeds to step ST45.

In step ST45, the processor determines whether or not the current time point tᵢ is the initial arrival time of the contrast agent on the basis of the index value dᵢ at the current time tᵢ and the maximum value dₘₐₓ of the past index values. Here, the current time point tᵢ is time point t₃, so the processor determines whether the current time point t₃ is the initial arrival time of the contrast agent on the basis of the index value d₃ at the current time point t₃ and the maximum value dₘₐₓ of past index values. Specifically, the processor compares the index value d₃ at the current time point t₃ with the maximum value dₘₐₓ of the past index values. Then, if the index value d₃ at the current time point t₃ exceeds the maximum value dₘₐₓ of the past index values (d₃>dₘₐₓ), the processor determines that the current time point t₃ is the initial arrival time of the contrast agent, proceeds to step ST46, and updates the initial arrival time TA of the contrast agent from time point t₁ to time point t₃. On the other hand, if the index value d₃ at the current time point t₃ does not exceed the maximum value dₘₐₓ of past index values (d₃≤dₘₐₓ), the processor determines that the current time point t₃ is not the initial arrival time of the contrast agent, and proceeds to step ST50 without updating the initial arrival time TA of the contrast agent.

Here, d₃=d₂=0, so d₃≤dₘₐₓ. Therefore, the processor determines that the number of pixels stained by the contrast agent has not increased rapidly, and proceeds to step ST50 without updating the initial arrival time TA of the contrast agent.

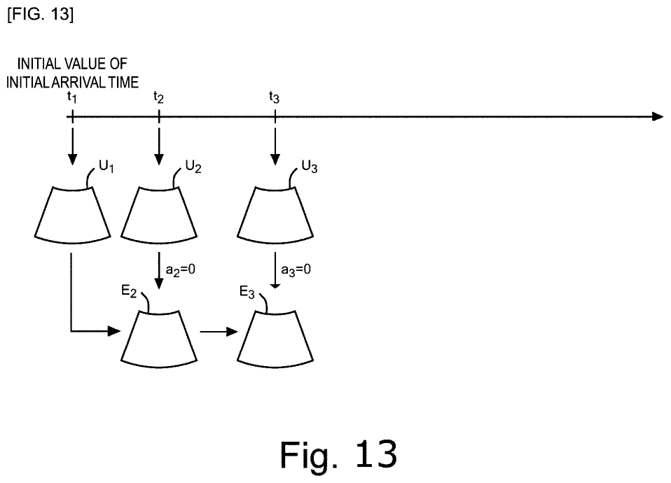

In step ST50, the processor creates a parametric image at time point t₃. FIG. 13 is an explanatory diagram of a method for creating a parametric image E₃ at time point t₃. At time point t₃, the total number a₃ of pixels exceeding the threshold value TH satisfies a₃=0. In this case, no pixels were stained with contrast agent, so the parametric image E₂ at time point t₂ is displayed as is as the parametric image E₃ at the current time point t₃. Once the parametric image E₃ is displayed, the process proceeds to step ST60.

In step ST60, whether or not the examination has ended is determined. Herein, the examination has not yet ended, and thus the process proceeds to step ST49.

In step ST49, the processor increments i. Herein, i=3, and thus i is incremented to 4. The process then returns to step ST30.

In step ST30, a processor determines whether or not the ultrasound image Uᵢ has been acquired at the current time point tᵢ. Herein, i has been incremented to i=4, and therefore, it is determined whether or not an ultrasound image U₄ has been acquired at the current time point t₄. If it is determined that the ultrasound image U₄ has been acquired at the current time point t₄, the process proceeds to step ST40.

In step ST40, a process of detecting the arrival time of the contrast agent is executed on the basis of the ultrasound image U₄ at the current time point t₄.

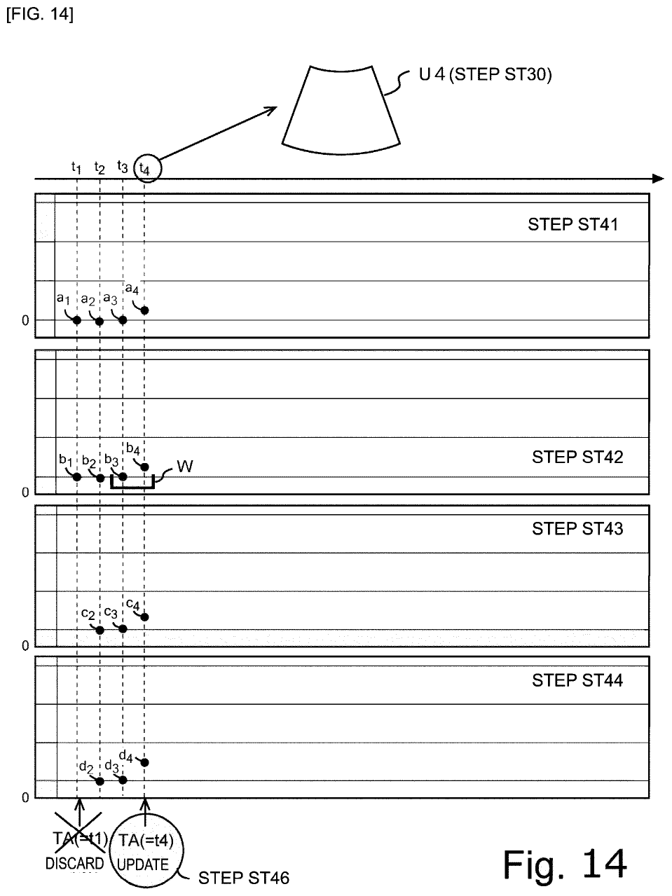

FIG. 14 is an explanatory diagram of step ST40 (steps ST41 to ST47) at time point t₄.

In step ST41, the processor determines the total number a₄ of pixels at the time point t₄. The method for calculating the total number of pixels is the same as the described method with reference to FIG. 9. Here, it is assumed that a₄>a₁ to a₃. Therefore, the ultrasound image U₄ at time point t₄ includes pixels whose pixel values exceed the threshold value Th. Once the total number a₄ of pixels has been calculated, the process proceeds to step ST42.

In step ST42, the processor executes a peak hold on the total number a₄ of pixels at the current time point t₄ to determine a peak value b₄. Specifically, the peak value b₄ is determined as follows.

The processor compares the total number a₄ of pixels obtained for the ultrasound image U₄ at the current time point t₄ with the maximum value aₘₐₓ of the total number of pixels obtained in the past. Furthermore, if the total number a₄ of pixels obtained for the ultrasound image U₄ at the current time point t₄ is greater than the maximum value aₘₐₓ of the total number of pixels obtained in the past (a₄>aₘₐₓ), the processor determines the total number a₄ of pixels obtained for the ultrasound image U₄ at the current time point t₄ as the peak value b₄. On the other hand, if the total number a₄ of pixels obtained for the ultrasound image U₄ at the current time point t₄ is at the maximum value aₘₐₓ or less of the total number of pixels obtained in the past (a₄≤aₘₐₓ), the peak value b₃ at the time point t₃ is retained as the peak value b₄ at the current time point t₄.

Herein, the total number a₄ of pixels at the time point t₄ is larger than the maximum value aₘₐₓ of the total number of pixels obtained in the past (i.e., larger than a₁ to a₃). Therefore, a₄>aₘₐₓ, so the processor determines the total number a₄ of pixels obtained for the ultrasound image U₄ at the current time point t₄ as the peak value b₄. Therefore, the peak value b₄ is determined to satisfy b₄=a₄ (>0). After the peak value b₄ is determined, the process proceeds to step ST43.

In step ST43, the processor calculates the change amount c₄ of the peak value at time point t₄. Specifically, the processor sets the time window W including two pieces of data (i.e., a time window including the peak value b₄ at the current time point t₄ and the peak value b₃ at the time point t₃ immediately preceding the current time point t₄), and calculates the change amount c₄ between the two pieces of data b₃ and b₄ included in the time window W. The change amount c₄ can be calculated using the following equation.

$${c}_{4} = ( {b}_{4} - {b}_{3} ) / ( {t}_{4} - {t}_{3} )$$

After calculating the change amount c₄ at time point t₄, the process proceeds to step ST44.

In step ST44, the index value d₄ at the current time point t₄ is calculated. The index value d₄ at the current time point t₄ can be calculated by d₄=c₄/b₄. After calculating the index value d₄, the process proceeds to step ST45.

In step ST45, the processor determines whether or not the current time point t₄ is the initial arrival time of the contrast agent on the basis of the index value d₄ at the current time t₄ and the maximum value dₘₐₓ of the past index values. Specifically, the processor compares the index value d₄ at the current time point t₄ with the maximum value dₘₐₓ of the past index values. Then, if the index value d₄ at the current time point t₄ exceeds the maximum value dₘₐₓ of the past index values (d₄>dₘₐₓ), the processor determines that the current time point t₄ is the initial arrival time of the contrast agent, proceeds to step ST46, and updates the initial arrival time TA of the contrast agent from time point t₁ to time point t₄. On the other hand, if the index value d₄ at the current time point t₄ does not exceed the maximum value dₘₐₓ of past index values (d₄≤dₘₐₓ), the processor determines that the current time point t₃ is not the initial arrival time of the contrast agent, and proceeds to step ST50 without updating the initial arrival time TA of the contrast agent.

Here, d₄>dₘₐₓ, so the process proceeds to step ST46, where the processor discards the previously determined initial arrival time TA (=t₁) and updates the initial arrival time TA of the contrast agent from time point t₁ to time point t₄. The process then proceeds to step ST48.

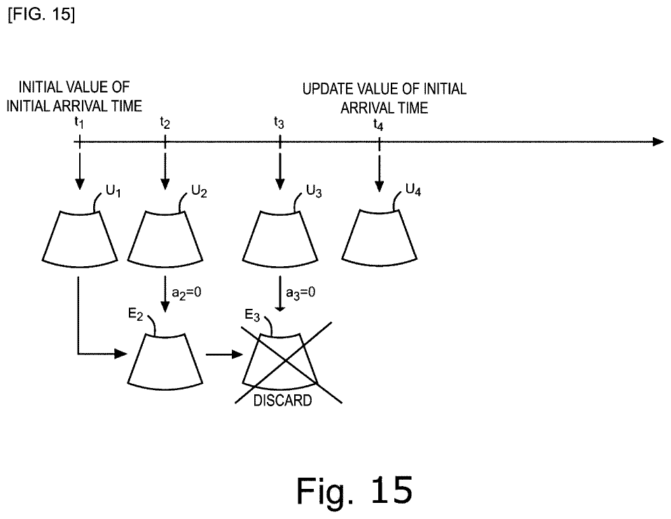

FIG. 15 is an explanatory diagram of step ST48.

The processor discards the parametric image E₃ and sets the ultrasound image U₄ at time point t₄ as the initial image of the parametric images. After the initial image of the parametric image is set, the process proceeds to step ST49.

In step ST49, the processor increments i. Here, i=4, and thus i is incremented to 5. The process then returns to step ST30.

In step ST30, a processor determines whether or not the ultrasound image Uᵢ has been acquired at the current time point tᵢ. Herein, i has been incremented to i=5, and therefore, it is determined whether or not an ultrasound image U₅ has been acquired at the current time point t₅. If it is determined that the ultrasound image U₅ has been acquired at the current time point t₅, the process proceeds to step ST40.

In step ST40, a process of detecting the arrival time of the contrast agent is executed on the basis of the ultrasound image U₅ at the current time point t₅.

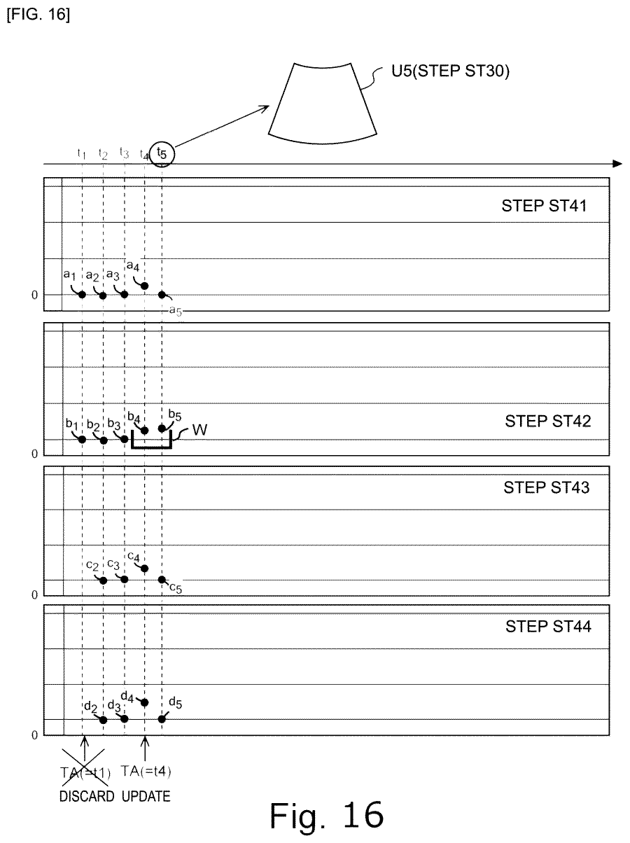

FIG. 16 is an explanatory diagram of step ST40 (steps ST41 to ST47) at time point t₅.

In step ST41, the processor determines the total number a₅ of pixels at the time point t₅. The method for calculating the total number of pixels is the same as the described method with reference to FIG. 9. Here, it is assumed that a₅=0. Once the total number a₅ of pixels has been calculated, the process proceeds to step ST42.

In step ST42, the processor executes a peak hold on the total number a₅ of pixels at the current time point t₅ to determine a peak value b₅. Specifically, the peak value b₅ is determined as follows.

The processor compares the total number a₅ of pixels obtained for the ultrasound image U s at the current time point t₅ with the maximum value aₘₐₓ of the total number of pixels obtained in the past. Furthermore, if the total number a₅ of pixels obtained for the ultrasound image U s at the current time point t₅ is greater than the maximum value aₘₐₓ of the total number of pixels obtained in the past (a₅>aₘₐₓ), the processor determines the total number a₅ of pixels obtained for the ultrasound image U s at the current time point t₅ as the peak value b₅. On the other hand, if the total number a₅ of pixels obtained for the ultrasound image U s at the current time point t₅ is at the maximum value aₘₐₓ or less of the total number of pixels obtained in the past (a₅≤aₘₐₓ), the peak value b₄ of the immediately preceding time point t₄ is retained as the peak value b₅ of the current time point t₅.

Here, the maximum value aₘₐₓ of the total number of pixels obtained in the past is aₘₐₓ=a₄, and the total number of pixels as at time point t₅ satisfies a₅=0. Therefore, since a₅≤aₘₐₓ, the processor holds the peak value b₄ at the immediately previous time point t₄ as the peak value b₅ at the current time point t₅, or in other words, determines that b₅=b₄. After the peak value b₅ is determined, the process proceeds to step ST43.

In step ST43, the processor calculates the change amount c₅ of the peak value at time point t₅. Specifically, the processor sets the time window W including two pieces of data (i.e., a time window including the peak value b₅ at the current time point t₅ and the peak value b₄ at the time point t₄ immediately preceding the current time point t₅), and calculates the change amount c₅ between the two pieces of data b₄ and b₅ included in the time window W. The change amount c₄ can be calculated using the following equation.

$${c}_{5} = ( {b}_{5} - {b}_{4} ) / ( {t}_{5} - {t}_{4} )$$

Since b₅=b₄, c₅=0 is calculated. After calculating the change amount c₅ at time point t₅, the process proceeds to step ST44.

In step ST44, the index value d₅ at the current time point t₅ is calculated. The index value d₅ at the current time point t₅ can be calculated by d₅=c₅/b₅. Since c₅=0, d₅=0 is calculated. After calculating the index value d₅, the process proceeds to step ST45.

In step ST45, the processor determines whether or not the current time point t₅ is the initial arrival time of the contrast agent on the basis of the index value d₅ at the current time t₅ and the maximum value dₘₐₓ of the past index values. Specifically, the processor compares the index value d₅ at the current time point t₅ with the maximum value dₘₐₓ of the past index values. Then, if the index value d₅ at the current time point t₅ exceeds the maximum value dₘₐₓ of the past index values (d₅>dₘₐₓ), the processor determines that the current time point t₅ is the initial arrival time of the contrast agent, proceeds to step ST46, and updates the initial arrival time TA of the contrast agent from time point t₄ to time point t₅. On the other hand, if the index value d₅ at the current time point t₅ does not exceed the maximum value dₘₐₓ of past index values (d₅≤dₘₐₓ), the processor determines that the current time point t₅ is not the initial arrival time of the contrast agent, and proceeds to step ST50 without updating the initial arrival time TA of the contrast agent.

Here, the maximum value dₘₐₓ of the past index values is dₘₐₓ=d₄, and the index value d₅ at time point t₅ satisfies d₅=0. Therefore, d₅≤dₘₐₓ, so the processor determines not to update the initial arrival time TA of the contrast agent, but to maintain the previously determined initial arrival time TA (=t₄), and proceeds to step ST50.

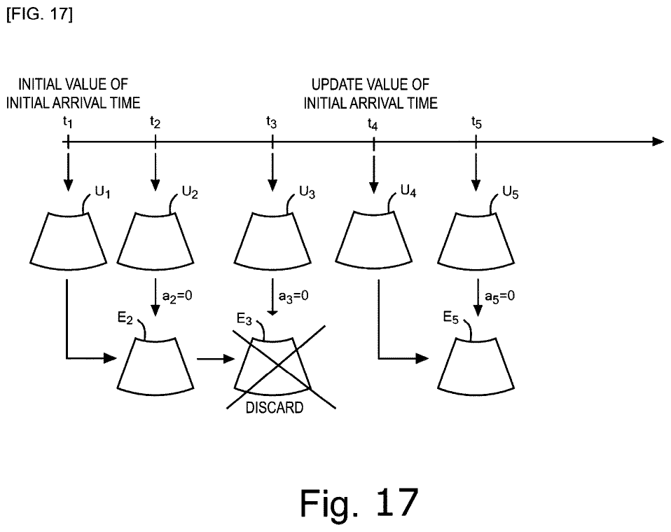

In step ST50, the processor creates a parametric image at time point t₅. FIG. 17 is an explanatory diagram of a method for creating a parametric image E₅ at time point t₅. At time point t₅, the total number a₅ of pixels exceeding the threshold value TH satisfies a₅=0. In this case, there are no pixels stained with contrast agent, so the ultrasound image U₄ at time point t₄ is displayed as is as the parametric image E₅ at the current time point t₅. Once the parametric image E₅ is displayed, the process proceeds to step ST60.

In step ST60, whether or not the examination has ended is determined. Herein, the examination has not yet ended, and thus the process proceeds to step ST49.

In step ST49, the processor increments i. Herein, i=5, and thus i is incremented to 6. The process then returns to step ST30.

In step ST30, a processor determines whether or not the ultrasound image Uᵢ has been acquired at the current time point tᵢ. Herein, i has been incremented to i=6, and therefore, it is determined whether or not an ultrasound image U₆ has been acquired at the current time point t₆. If it is determined that the ultrasound image U₃ has been acquired at the current time point t₆, the process proceeds to step ST40.

In step ST40, a process of detecting the arrival time of the contrast agent is executed on the basis of the ultrasound image U₃ at the current time point t₆.

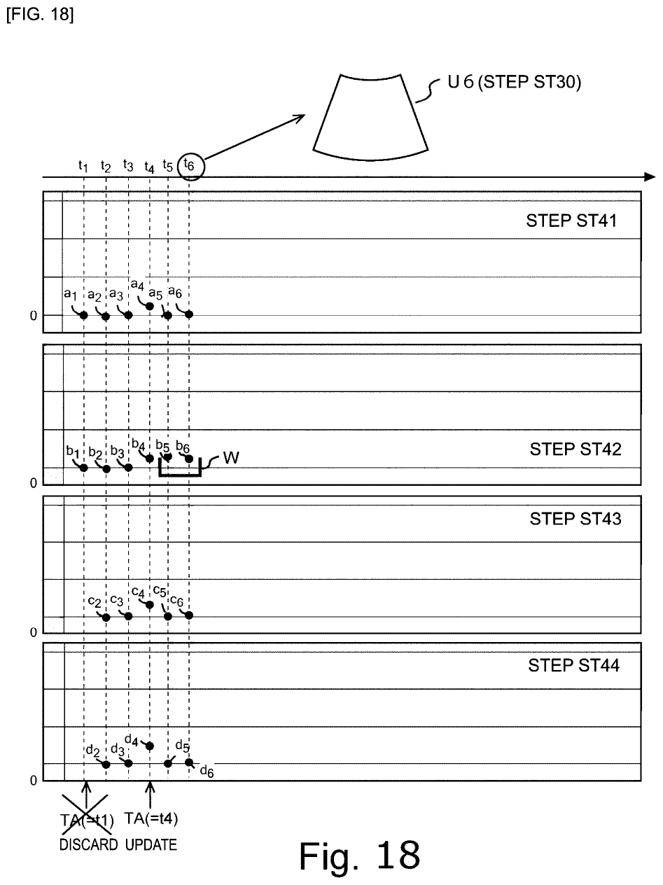

FIG. 18 is an explanatory diagram of step ST40 (steps ST41 to ST47) at time point t₆.

In step ST41, the processor determines the total number a₆ of pixels at the time point t₆. The method for calculating the total number of pixels is the same as the described method with reference to FIG. 9. Here, it is assumed that a₆=0. Once the total number a₆ of pixels has been calculated, the process proceeds to step ST42.

In step ST42, the processor executes a peak hold on the total number a₆ of pixels at the current time point t₆ to determine a peak value b₆. Specifically, the peak value b₆ is determined as follows.

The processor compares the total number a₆ of pixels obtained for the ultrasound image U₆ at the current time point t₆ with the maximum value aₘₐₓ of the total number of pixels obtained in the past. Furthermore, if the total number a₆ of pixels obtained for the ultrasound image U₆ at the current time point t₆ is greater than the maximum value aₘₐₓ of the total number of pixels obtained in the past (a₆>aₘₐₓ), the processor determines the total number a₆ of pixels obtained for the ultrasound image U₆ at the current time point t₆ as the peak value b₆. On the other hand, if the total number a₆ of pixels obtained for the ultrasound image U₆ at the current time point t₆ is at the maximum value aₘₐₓ or less of the total number of pixels obtained in the past (a₆≤aₘₐₓ), the peak value b₅ at the immediately preceding time point t₅ is retained as the peak value b₆ at the current time point t₆.

Here, the maximum value aₘₐₓ of the total number of pixels obtained in the past is aₘₐₓ=a₄, and the total number of pixels a₆ at the current time point t₆ satisfies a₆=0. Therefore, since a₆≤aₘₐₓ, the processor holds the peak value b₅ at the immediately previous time point t₅ as the peak value b₆ at the current time point t₆, or in other words, determines that b₆=b₆. After the peak value b₆ is determined, the process proceeds to step ST43.

In step ST43, the processor calculates the change amount c₆ of the peak value at time point t₆. Specifically, the processor sets the time window W including two pieces of data (i.e., a time window including the peak value b₆ at the current time point t₆ and the peak value b₅ at the time point t₅ immediately preceding the current time point t₆), and calculates the change amount c₆ between the two pieces of data b₅ and b₆ included in the time window. Here, b₅=b₆, so the change amount c₆ satisfies c₆=0. After calculating the change amount c₆, the process proceeds to step ST44.

In step ST44, the index value d₆ at the current time point t₆ is calculated. The index value d₆ at the current time point t₆ can be calculated by d₆=c₆/b₆. Here, c₆=0, so d₆=0. After calculating the index value d₆, the process proceeds to step ST45.

In step ST45, the processor determines whether or not the current time point t₆ is the initial arrival time of the contrast agent on the basis of the index value d₆ at the current time point t₆ and the maximum value dₘₐₓ of the past index values. Specifically, the processor compares the index value d₆ at the current time point t₆ with the maximum value dₘₐₓ of the past index values. Then, if the index value d₆ at the current time point t₆ exceeds the maximum value dₘₐₓ of the past index values (_{d}>dₘₐₓ), the processor determines that the current time point t₆ is the initial arrival time of the contrast agent, proceeds to step ST46, and updates the initial arrival time TA of the contrast agent from time point t₄ to time point t₆. On the other hand, if the index value d₆ at the current time point t₆ does not exceed the maximum value dₘₐₓ of past index values (d₆≤dₘₐₓ), the processor determines that the current time point t₆ is not the initial arrival time of the contrast agent, and proceeds to step ST50 without updating the initial arrival time TA of the contrast agent.

Here, the maximum value dₘₐₓ of the past index values is dₘₐₓ=d₄, and the index value d₆ at the current time point t₆ satisfies d₆=0. Therefore, d₆≤dₘₐₓ, so the processor determines not to update the initial arrival time TA of the contrast agent, but to maintain the previously determined initial arrival time TA (=t₄), and proceeds to step ST50.

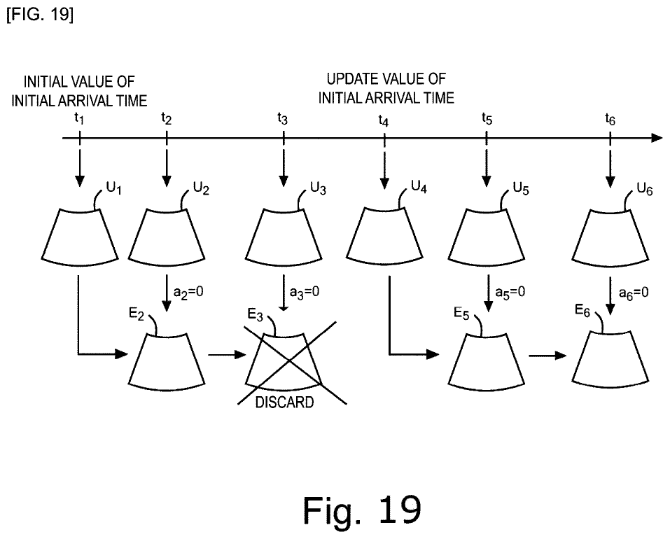

In step ST50, the processor creates a parametric image at time point t₆. FIG. 19 is an explanatory diagram of a method for creating a parametric image E₆ at time point t₆. At time point t₆, the total number a₆ of pixels exceeding the threshold value TH satisfies a₆=0. In this case, no pixels were stained with contrast agent, so the parametric image E₅ at time point t₅ is displayed as is as the parametric image E₆ at the current time point t₆. Once the parametric image E₆ is displayed, the process proceeds to step ST60.

In step ST60, whether or not the examination has ended is determined. Herein, the examination has not yet ended, and thus the process proceeds to step ST48.

In step ST48, the processor increments i. Here, i=6, and thus i is incremented to 7. The process then returns to step ST30.

In step ST30, a processor determines whether or not the ultrasound image Uᵢ has been acquired at the current time point tᵢ. Herein, i has been incremented to i=7, and therefore a determination is made as to whether or not an ultrasound image U₇ has been acquired at the current time point t₇. If it is determined that the ultrasound image U₇ has been acquired at the current time point t₇, the process proceeds to step ST40.

In step ST40, a process of detecting the arrival time of the contrast agent is executed on the basis of the ultrasound image U₇ at the current time point t₇.

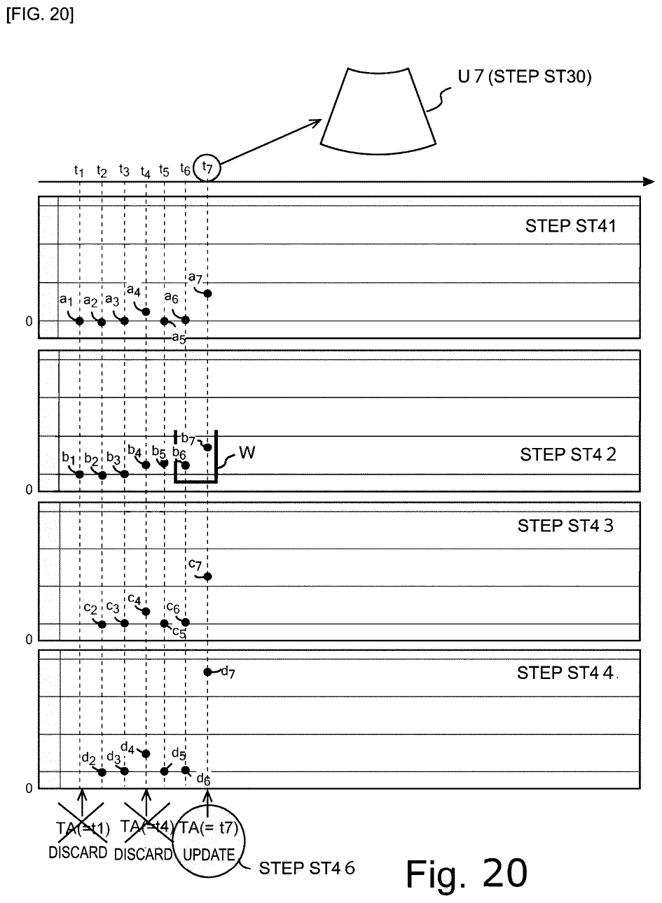

FIG. 20 is an explanatory diagram of step ST40 (steps ST41 to ST47) at time point t₇.

In step ST41, the processor determines the total number a₇ of pixels at the time point t₇. The method for calculating the total number of pixels is the same as the described method with reference to FIG. 9. Here, it is assumed that a₇ is a larger value than a₁ to a₆. Once the total number a₇ of pixels has been calculated, the process proceeds to step ST42.

In step ST42, the processor executes a peak hold on the total number a₇ of pixels at the current time point t₇ to determine a peak value b₇. Specifically, the peak value b₇ is determined as follows.

The processor compares the total number a₇ of pixels obtained for the ultrasound image U₇ at the current time point t₇ with the maximum value aₘₐₓ of the total number of pixels obtained in the past. Furthermore, if the total number a₇ of pixels obtained for the ultrasound image U₇ at the current time point t₇ is greater than the maximum value aₘₐₓ of the total number of pixels obtained in the past (a₇>aₘₐₓ), the processor determines the total number a₇ of pixels obtained for the ultrasound image U₇ at the current time point t₇ as the peak value b₇. On the other hand, if the total number a₇ of pixels obtained for the ultrasound image U₇ at the current time point t₇ is at the maximum value aₘₐₓ or less of the total number of pixels obtained in the past (a₇≤aₘₐₓ), the peak value b₆ at the time point t₆ is retained as the peak value b₇ at the current time point t₇.

Here, the maximum value aₘₐₓ of the total number of pixels obtained in the past is aₘₐₓ=a₄, and the total number of pixels a₇ at the current time point t₇ satisfies a₇>a₄. Therefore, a₇>aₘₐₓ, so the processor determines the total number a₇ of pixels obtained for the ultrasound image U₇ at the current time point t₇ as the peak value b₇. Therefore, the peak value b₇ is determined as b₇=a₇. b₇ is a value greater than b₆ (b₇>b₆). After the peak value b₇ is determined, the process proceeds to step ST43.

In step ST43, the processor calculates the change amount c₇ of the peak value at time point t₇. Specifically, the processor sets the time window W including two pieces of data (i.e., a time window including the peak value b₇ at the current time point t₇ and the peak value b₆ at the time point t₆ immediately preceding the current time point t₇), and calculates the change amount c₇ between the two pieces of data b₆ and b₇ included in the time window. The change amount c₇ can be expressed by the following formula.

c₇=(b₇−b₆)/(t₇−t₆)

After calculating the change amount c₇ of the peak value, the process proceeds to step ST44.

In step ST44, the index value d₇ at the current time point t₇ is calculated. The index value d₇ at the current time point t₇ can be calculated by d₇=c₇/b₇. After calculating the index value d₄, the process proceeds to step ST45.

In step ST45, the processor determines whether or not the current time point t₇ is the initial arrival time of the contrast agent on the basis of the index value d₇ at the current time point t₇ and the maximum value dₘₐₓ of the past index values. Specifically, the processor compares the index value d₇ at the current time point t₇ with the maximum value dₘₐₓ of the past index values. Then, if the index value d₇ at the current time point t₇ exceeds the maximum value dₘₐₓ of the past index values (d₇>dₘₐₓ), the processor determines that the current time point t₇ is the initial arrival time of the contrast agent, proceeds to step ST46, and updates the initial arrival time TA of the contrast agent from time point t₄ to time point t₇. On the other hand, if the index value d₇ at the current time point t₇ does not exceed the maximum value dₘₐₓ of past index values (d₇≤dₘₐₓ), the processor determines that the current time point t₇ is not the initial arrival time of the contrast agent, and proceeds to step ST50 without updating the initial arrival time TA of the contrast agent.

Here, the maximum value dₘₐₓ of the past index values is dₘₐₓ=d₄, and the index value d₇ at the current time point t₇ satisfies d₇>d₄. However, d₇>dₘₐₓ, so the process proceeds to step ST46, where the processor discards the previously determined initial arrival time TA (=t₄) and updates the initial arrival time TA of the contrast agent from time point t₄ to time point t₇. The process then proceeds to step ST48.

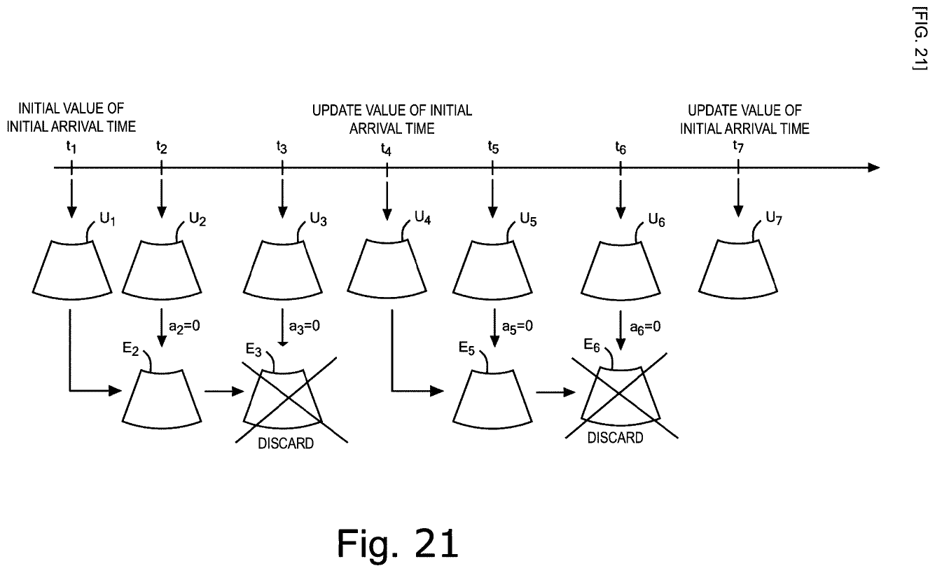

FIG. 21 is an explanatory diagram of step ST48.

The processor discards the parametric image E₆ and sets the ultrasound image U₇ at time point t₇ as the initial image of the parametric images. After the initial image of the parametric image is set, the process proceeds to step ST49.

In step ST49, the processor increments i. Herein, i=7, and thus i is incremented to 8. The process then returns to step ST30.

In step ST30, a processor determines whether or not the ultrasound image Uᵢ has been acquired at the current time point tᵢ. Herein, i has been incremented to i=8, and therefore, it is determined whether or not an ultrasound image U₈ has been acquired at the current time point t₈. If it is determined that the ultrasound image U₈ has been acquired at the current time point t₈, the process proceeds to step ST40.

In step ST40, a process of detecting the arrival time of the contrast agent is executed on the basis of the ultrasound image U₈ at the current time point t₈.

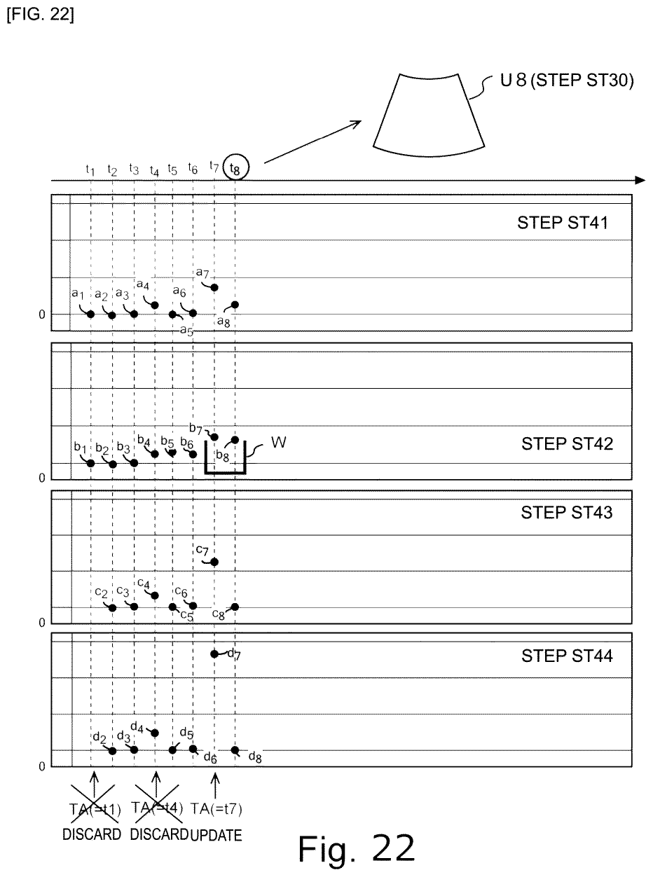

FIG. 22 is an explanatory diagram of step ST40 (steps ST41 to ST47) at time point t₈.

In step ST41, the processor determines the total number a₈ of pixels at the time point t₈. The method for calculating the total number of pixels is the same as the described method with reference to FIG. 9. Here, it is assumed that a₈<a₇. Once the total number a₈ of pixels has been calculated, the process proceeds to step ST42.

In step ST42, the processor executes a peak hold on the total number a₈ of pixels at the current time point t₈ to determine a peak value b₈. Specifically, the peak value b₈ is determined as follows.

The processor compares the total number a₈ of pixels obtained for the ultrasound image U₈ at the current time point t₈ with the maximum value aₘₐₓ of the total number of pixels obtained in the past. Furthermore, if the total number a₈ of pixels obtained for the ultrasound image U₈ at the current time point t₈ is greater than the maximum value aₘₐₓ of the total number of pixels obtained in the past (a₈>aₘₐₓ), the processor determines the total number a₈ of pixels obtained for the ultrasound image U₈ at the current time point t₈ as the peak value b₈. On the other hand, if the total number a₈ of pixels obtained for the ultrasound image U₈ at the current time point t₈ is at the maximum value aₘₐₓ or less of the total number of pixels obtained in the past (a₈≤aₘₐₓ), the peak value b₇ of the immediately preceding time point t₇ is retained as the peak value b₈ of the current time point t₈.

Here, the maximum value aₘₐₓ of the total number of pixels obtained in the past is aₘₐₓ=a₇, and the total number of pixels a₈ at time point t₈ satisfies a₈≤a₇. Therefore, since a₈≤aₘₐₓ, the processor holds the peak value b₇ at the immediately previous time point t₇ as the peak value b₈ at the current time point t₈, or in other words, determines that b₈=b₇. After the peak value b₈ is determined, the process proceeds to step ST43.

In step ST43, the processor calculates the change amount c₈ of the peak value at time point t₈. Specifically, the processor sets the time window W including two pieces of data (i.e., a time window including the peak value b₈ at the current time point t₈ and the peak value b₇ at the time point t₇ immediately preceding the current time point t₈), and calculates the change amount c₈ between the two pieces of data b₇ and b₈ included in the time window W. The change amount c₈ can be expressed by the following formula.

$${c}_{8} = ( {b}_{8} - {b}_{7} ) / ( {t}_{8} - {t}_{7} )$$

Since b₈=b₇, c₈=0 is calculated. After calculating the change amount c₈ at time point t₈, the process proceeds to step ST44.

In step ST44, the index value d₈ at the current time point t₈ is calculated. The index value d₈ at the current time point t₈ can be calculated by d₈=c₈/b₈. Since c₈=0, d₈=0 is calculated. After calculating the index value d₈, the process proceeds to step ST45.

In step ST45, the processor determines whether or not the current time point t₈ is the initial arrival time of the contrast agent on the basis of the index value d₈ at the current time point t₈ and the maximum value dₘₐₓ of the past index values. Specifically, the processor compares the index value d₈ at the current time point t₈ with the maximum value dₘₐₓ of the past index values. Then, if the index value d₈ at the current time point t₈ exceeds the maximum value dₘₐₓ of the past index values (d₈>dₘₐₓ), the processor determines that the current time point t₈ is the initial arrival time of the contrast agent, proceeds to step ST46, and updates the initial arrival time TA of the contrast agent from time point t₇ to time point t₈. On the other hand, if the index value d₈ at the current time point t₈ does not exceed the maximum value dₘₐₓ of past index values (d₈≤dₘₐₓ), the processor determines that the current time point t₈ is not the initial arrival time of the contrast agent, and proceeds to step ST50 without updating the initial arrival time TA of the contrast agent.

Here, the maximum value dₘₐₓ of the past index values satisfies dₘₐₓ=d₇, and the index value d₈ at time point t₈ satisfies d₈<d₇. Therefore, d₈≤dₘₐₓ, so the processor determines not to update the initial arrival time TA of the contrast agent, but to maintain the previously determined initial arrival time TA (=t₇), and proceeds to step ST50.

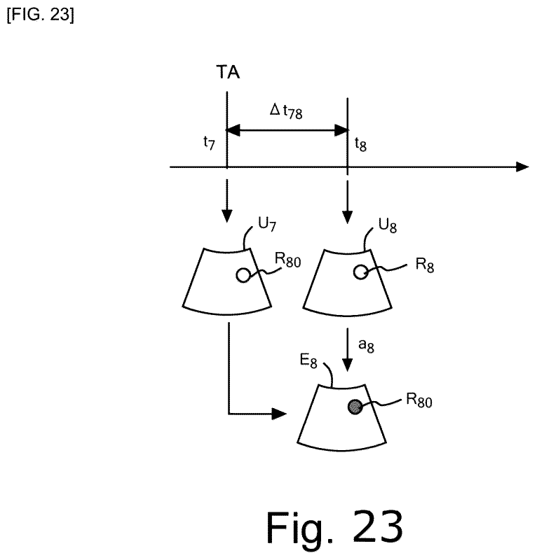

In step ST50, the processor creates a parametric image at time point t₈. FIG. 23 is an explanatory diagram of a method for creating a parametric image E₈ at time point t₈. At time point t₈, the total number of pixels exceeding the threshold value TH satisfies a₈>0. Note that in FIG. 23, for convenience of description, it is assumed that at time point t₈, pixels exceeding the threshold value TH (total number of pixels a₈) appear to be concentrated in a region R8 of an ultrasound image U₈. Therefore, the processor identifies a region R80 corresponding to region R8 in ultrasound image U₇, which is the reference image for the parametric image, and assigns a color to the pixels of region R80 corresponding to the time difference Δt₇₈ between time point t₇ (initial arrival time TA of the contrast agent) and time point t₈, thereby creating a parametric image E₈ at time point t₈. Once the parametric image E₈ is displayed, the process proceeds to step ST60.

In step ST60, whether or not the examination has ended is determined. Herein, the examination has not yet ended, and thus the process proceeds to step ST48.

In step ST48, the processor increments i. Here, i=8, and thus i is incremented to 9. The process then returns to step ST30.

In step ST30, a processor determines whether or not the ultrasound image Uᵢ has been acquired at the current time point tᵢ. Herein, i has been incremented to i=9, and therefore, it is determined whether or not an ultrasound image U₉ has been acquired at the current time point t₉. If it is determined that the ultrasound image U₉ has been acquired at the current time point t₉, the process proceeds to step ST40.

In step ST40, a process of detecting the arrival time of the contrast agent is executed on the basis of the ultrasound image U₉ at the current time point t₉.

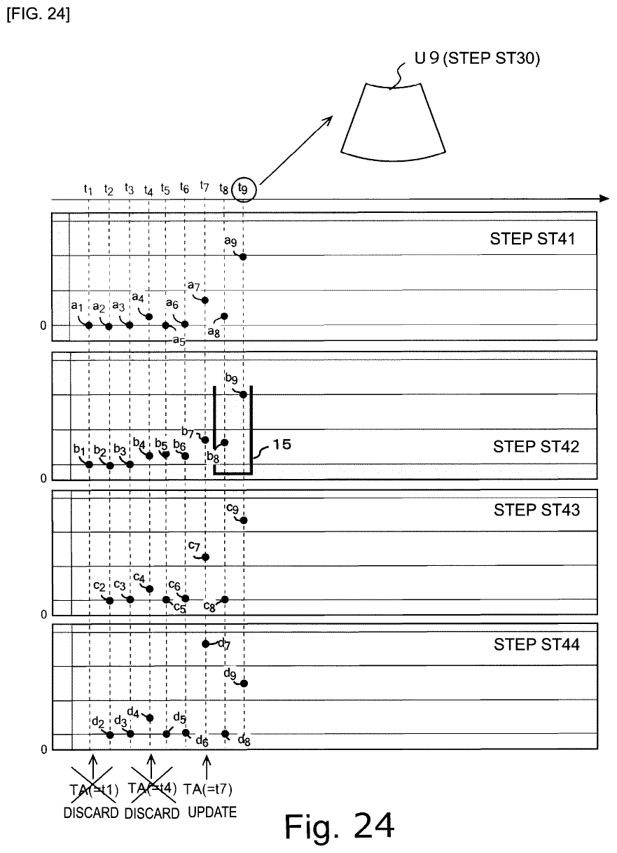

FIG. 24 is an explanatory diagram of step ST40 (steps ST41 to ST47) at time point t₉.

In step ST41, the processor determines the total number a₉ of pixels at the time point t₉. The method for calculating the total number of pixels is the same as the described method with reference to FIG. 9. Here, it is assumed that a₉>a₁ to a₈. Once the total number a₉ of pixels has been calculated, the process proceeds to step ST42.

In step ST42, the processor executes a peak hold on the total number a₉ of pixels at the current time point t₉ to determine a peak value b₉. Specifically, the peak value b₉ is determined as follows.

The processor compares the total number a₉ of pixels obtained for the ultrasound image U₉ at the current time point t₉ with the maximum value aₘₐₓ of the total number of pixels obtained in the past. Furthermore, if the total number a₉ of pixels obtained for the ultrasound image U₉ at the current time point t₉ is greater than the maximum value aₘₐₓ of the total number of pixels obtained in the past (a₉>aₘₐₓ), the processor determines the total number a₉ of pixels obtained for the ultrasound image U₉ at the current time point t₉ as the peak value b₉. On the other hand, if the total number a₉ of pixels obtained for the ultrasound image U₉ at the current time point t₉ is at the maximum value aₘₐₓ or less of the total number of pixels obtained in the past (a₉≤aₘₐₓ), the peak value b₈ at the immediately preceding time point t₈ is retained as the peak value b₉ at the current time point t₉.

Here, the maximum value aₘₐₓ of the total number of pixels obtained in the past is aₘₐₓ=a₇, and the total number of pixels a₉ at the current time point t₉ satisfies a₉>a₇. Therefore, a₉>aₘₐₓ, so the processor determines the total number of pixels a₉ at the current time point t₉ as the peak value b_{g} of the total number of pixels at the current time point t₉. Therefore, the peak value b₉ is determined to satisfy b₉=a₉ (>b₈). After the peak value b₉ is determined, the process proceeds to step ST43.

In step ST43, the processor calculates the change amount c₉ of the peak value at time point t₉. Specifically, the processor sets the time window W including two pieces of data (i.e., a time window including the peak value b₉ at the current time point t₉ and the peak value b₈ at the time point t₈ immediately preceding the current time point t₉), and calculates the change amount c₉ between the two pieces of data b₈ and b_{g} included in the time window W. The change amount c₉ can be expressed by the following formula.

$${c}_{9} = ( {b}_{9} - {b}_{8} ) / ( {t}_{9} - {t}_{8} )$$

Here, c₉ is a value greater than c₇ (c₉>c₇). After calculating the change amount c₉, the process proceeds to step ST44.

In step ST44, the index value d₉ at the current time point t₉ is calculated. The index value d₉ at the current time point t₉ can be calculated by d₉=c₉/b₉. After calculating the index value dg, the process proceeds to step ST45.

In step ST45, the processor determines whether or not the current time point t₉ is the initial arrival time of the contrast agent on the basis of the index value d₉ at the current time point t₉ and the maximum value dₘₐₓ of the past index values. Specifically, the processor compares the index value d₉ at the current time point t₉ with the maximum value dₘₐₓ of the past index values. Then, if the index value d₉ at the current time point t₉ exceeds the maximum value dₘₐₓ of the past index values (d₉>dₘₐₓ), the processor determines that the current time point t₉ is the initial arrival time of the contrast agent, proceeds to step ST46, and updates the initial arrival time TA of the contrast agent from time point t₇ to time point t₉. On the other hand, if the index value d₉ at the current time point t₉ does not exceed the maximum value dₘₐₓ of past index values (d₉≤dₘₐₓ), the processor determines that the current time point t₉ is not the initial arrival time of the contrast agent, and proceeds to step ST50 without updating the initial arrival time TA of the contrast agent.

Here, the maximum index value dₘₐₓ obtained in the past satisfies dₘₐₓ=d₇, and the index value d₉ at time point t₉ satisfies d₉<d₇. Therefore, d₉≤dₘₐₓ, so the processor determines not to update the initial arrival time TA of the contrast agent, but to maintain the previously determined initial arrival time TA (=t₇), and proceeds to step ST50.

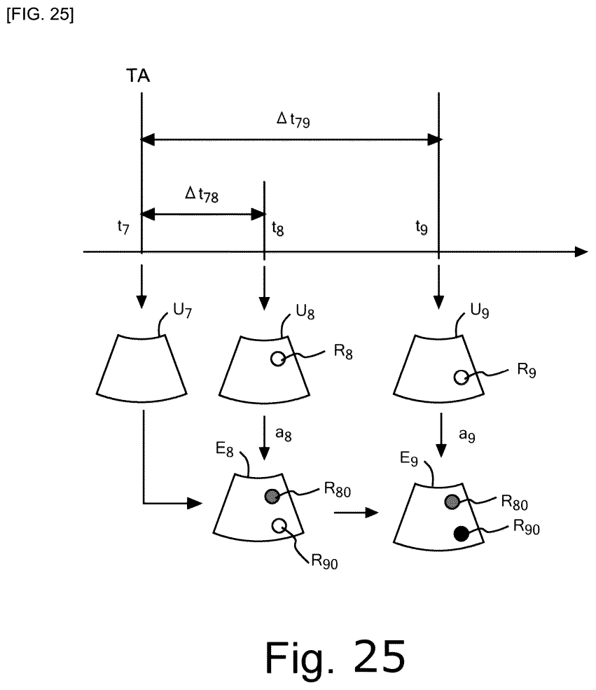

In step ST50, the processor creates a parametric image at time point t₉. FIG. 25 is an explanatory diagram of a method for creating a parametric image E₉ at time point t₉. At time point t₉, the total number of pixels exceeding the threshold value TH satisfies a₉>0. Note that in FIG. 25, for convenience of description, it is assumed that at time point t₉, pixels exceeding the threshold value TH (total number of pixels a₈) appear to be concentrated in a region R9 of an ultrasound image U₈. Therefore, the processor identifies a region R90 corresponding to region R9 in the parametric image E₈, and assigns a color to the pixels of region R90 corresponding to the time difference Δt₇₉ between time point t7 (initial arrival time TA of the contrast agent) and time point t₉, thereby creating a parametric image E₉ at time point t₉. Once the parametric image E₉ is displayed, the process proceeds to step ST60.

In step ST60, whether or not the examination has ended is determined. Herein, the examination has not yet ended, and thus the process proceeds to step ST48.

In step ST48, the processor increments i. Herein, i=9, and thus i is incremented to 10. The process then returns to step ST30.

In step ST30, a processor determines whether or not the ultrasound image Uᵢ has been acquired at the current time point tᵢ. Herein, i has been incremented to i=10, and therefore, it is determined whether or not an ultrasound image U₁₀ has been acquired at the current time point t₁₀. If it is determined that the ultrasound image U₁₀ has been acquired at the current time point t₁₀, the process proceeds to step ST40.

In step ST40, a process of detecting the arrival time of the contrast agent is executed on the basis of the ultrasound image U₁₀ at the current time point t₁₀.

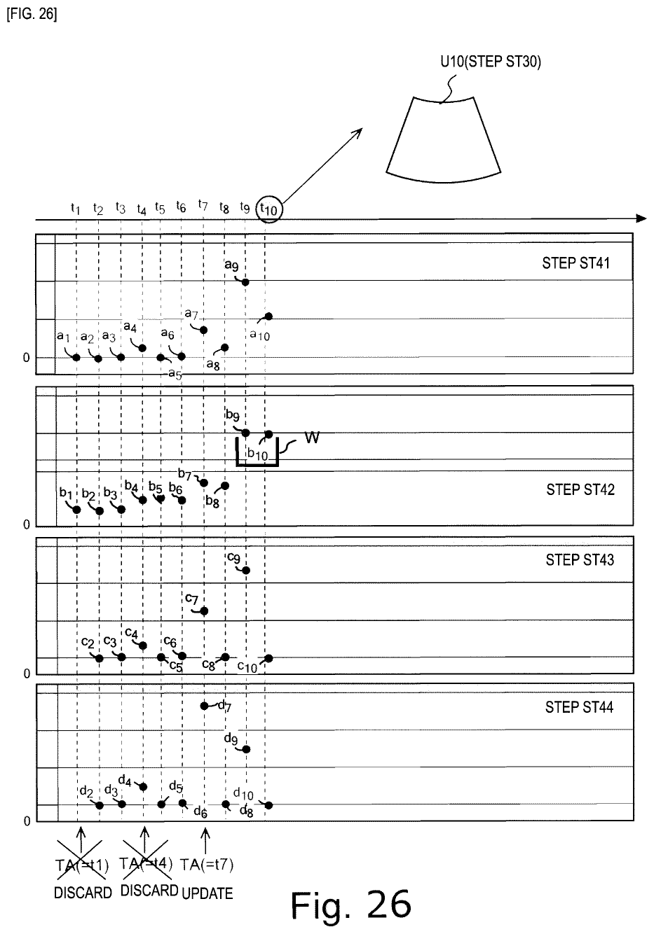

FIG. 26 is an explanatory diagram of step ST40 (steps ST41 to ST47) at time point t₁₀.

In step ST41, the processor determines the total number a₁₀ of pixels at the time point t₁₀. The method for calculating the total number of pixels is the same as the described method with reference to FIG. 9. Here, it is assumed that a₁₀<a₉. Once the total number a₁₀ of pixels has been calculated, the process proceeds to step ST42.

In step ST42, the processor executes a peak hold on the total number a₁₀ of pixels at the current time point t₁₀ to determine a peak value b₁₀. Specifically, the peak value b₁₀ is determined as follows.

The processor compares the total number a₁₀ of pixels obtained for the ultrasound image U₁₀ at the current time point t₁₀ with the maximum value aₘₐₓ of the total number of pixels of the past. Furthermore, if the total number a₁₀ of pixels obtained for the ultrasound image U₁₀ at the current time point t₁₀ is greater than the maximum value aₘₐₓ of the total number of pixels of the past (a₁₀>aₘₐₓ), the processor determines the total number a₁₀ of pixels obtained for the ultrasound image U₁₀ at the current time point t₁₀ as the peak value b₁₀. On the other hand, if the total number a₁₀ of pixels obtained for the ultrasound image U₁₀ at the current time point t₁₀ is at the maximum value aₘₐₓ or less of the total number of pixels obtained in the past (a₁₀≤aₘₐₓ), the peak value b₉ at the immediately preceding time point t₉ is retained as the peak value b₁₀ at the current time point t₁₀.

Here, the maximum value aₘₐₓ of the total number of pixels obtained in the past satisfies aₘₐₓ=a₉, and the total number of pixels a₁₀ at time point t₁₀ satisfies a₁₀<a₉. Therefore, a₁₀≤aₘₐₓ, and thus the processor holds the peak value be at time point t₉ as the peak value b₁₀ of the total number of pixels at the current time point t₁₀. Therefore, the peak value b₁₀ is determined to satisfy b₁₀=b₉. After the peak value b₁₀ is determined, the process proceeds to step ST43.

In step ST43, the processor calculates the change amount c₁₀ of the peak value at time point t₁₀. Specifically, the processor sets the time window W including two pieces of data (i.e., a time window including the peak value b₁₀ at the current time point t₁₀ and the peak value b₉ at the time point t₉ immediately preceding the current time point t₁₀), and calculates the change amount c₁₀ between the two pieces of data b₉ and b₁₀ included in the time window W. The change amount c₁₀ can be expressed by the following formula.

$${c}_{10} = ( {b}_{10} - {b}_{9} ) / ( {t}_{10} - {t}_{9} )$$

Here, b₁₀=b₉, so c₁₀=0. After calculating the change amount c₁₀ of the peak value, the process proceeds to step ST44.

In step ST44, the index value d₁₀ at the current time point t₁₀ is calculated. The index value d₁₀ at the current time point t₁₀ can be calculated by d₁₀=c₁₀/b₁₀. c₁₀=0, so d₁₀=0. After calculating the index value d₁₀, the process proceeds to step ST45.

In step ST45, the processor determines whether or not the current time point t₁₀ is the initial arrival time of the contrast agent on the basis of the index value d₁₀ at the current time point t₁₀ and the maximum value dₘₐₓ of the past index values. Specifically, the processor compares the index value d₁₀ at the current time point t₁₀ with the maximum value dₘₐₓ of the past index values. Then, if the index value d₁₀ at the current time point t₁₀ exceeds the maximum value dₘₐₓ of the past index values (d₁₀>dₘₐₓ), the processor determines that the current time point t₁₀ is the initial arrival time of the contrast agent, proceeds to step ST46, and updates the initial arrival time TA of the contrast agent from time point t₇ to time point t₁₀. On the other hand, if the index value d₁₀ at the current time point t₁₀ does not exceed the maximum value dₘₐₓ of past index values (d₁₀≤dₘₐₓ), the processor determines that the current time point t₁₀ is not the initial arrival time of the contrast agent, and proceeds to step ST50 without updating the initial arrival time TA of the contrast agent.

Here, the maximum index value dₘₐₓ obtained in the past satisfies dₘₐₓ=d₇, and the index value d₁₀ at time point t₁₀ satisfies d₁₀≤d₇. Therefore, d₁₀≤dₘₐₓ, so the processor determines not to update the initial arrival time TA of the contrast agent, but to maintain the previously determined initial arrival time TA (=t₇), and proceeds to step ST50.

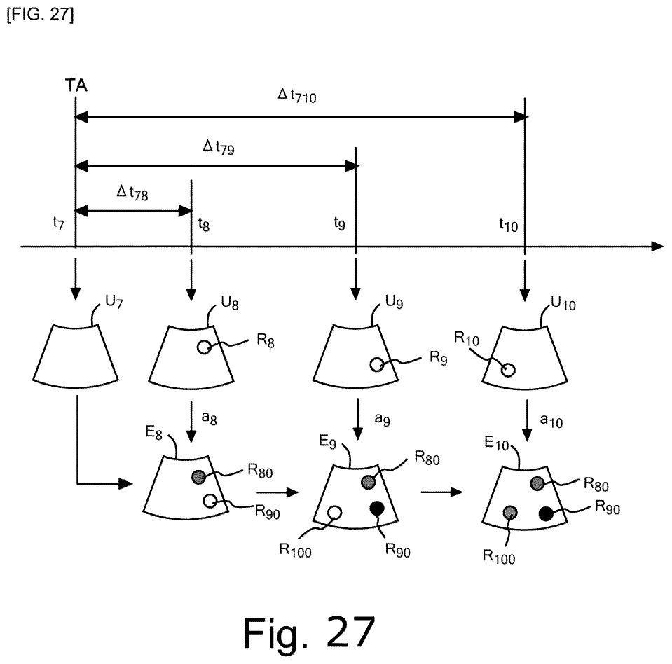

In step ST50, the processor creates a parametric image at time point t₁₀. FIG. 27 is an explanatory diagram of a method for creating a parametric image E₁₀ at time point t₁₀. Note that in FIG. 27, for convenience of description, it is assumed that at time point t₁₀, pixels exceeding the threshold value TH (total number of pixels as) appear to be concentrated in a region R10 of an ultrasound image U₈. Therefore, the processor identifies a region R100 corresponding to region R10 in the parametric image E₉, and assigns a color to the pixels of region R100 corresponding to the time difference Δt₇₁₀ between time point t₇ (initial arrival time TA of the contrast agent) and time point t₁₀, thereby creating a parametric image E₁₀ at time point t₁₀. Once the parametric image E₁₀ is displayed, the process proceeds to step ST60.

In step ST60, whether or not the examination has ended is determined. Herein, the examination has not yet ended, and thus the process proceeds to step ST48.

In step ST48, the processor increments i. Here, i=10, and thus i is incremented to 11. The process then returns to step ST30.

In step ST30, a processor determines whether or not the ultrasound image Uᵢ has been acquired at the current time point tᵢ. Herein, i has been incremented to i=11, and therefore, it is determined whether or not an ultrasound image U₁₁ has been acquired at the current time point t₁₁. If it is determined that the ultrasound image U₁₁ has been acquired at the current time point t₁₁, the process proceeds to step ST40.

In step ST40, a process of detecting the arrival time of the contrast agent is executed on the basis of the ultrasound image U n at the current time point t₁₁.

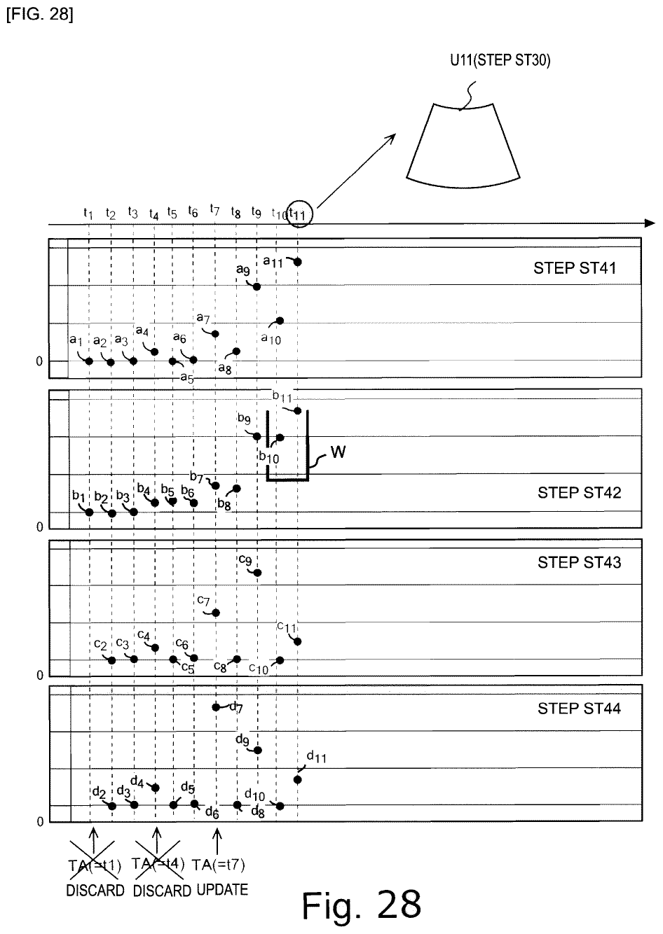

FIG. 28 is an explanatory diagram of step ST40 (steps ST41 to ST47) at time point t₁₁.

In step ST41, the processor determines the total number a₁₁ of pixels at the time point t₁₁. Once the total number a₁₁ of pixels has been calculated, the process proceeds to step ST42.

In step ST42, the processor executes a peak hold on the total number a₁₁ of pixels at the current time point t₁₁ to determine a peak value b₁₁. Specifically, the peak value b₁₁ is determined as follows.

The processor compares the total number a₁₁ of pixels obtained for the ultrasound image Uni at the current time point t₁₁ with the maximum value aₘₐₓ of the total number of pixels of the past. Furthermore, if the total number a₁₁ of pixels obtained for the ultrasound image U₁₁ at the current time point t₁₁ is greater than the maximum value aₘₐₓ of the total number of pixels of the past (a₁₁>aₘₐₓ), the processor determines the total number a₁₁ of pixels obtained for the ultrasound image U₁₁ at the current time point t₁₁ as the peak value b₁₁. On the other hand, if the total number a₁₁ of pixels obtained for the ultrasound image U₁₁ at the current time point t₁₁ is at the maximum value aₘₐₓ or less of the total number of pixels obtained in the past (a₁₁≤aₘₐₓ), the peak value b₁₀ at the immediately preceding time point t₁₀ is retained as the peak value b₁₁ at the current time point t₁₁.

Here, the maximum value aₘₐₓ of the total number of pixels obtained in the past is aₘₐₓ=a₉, and the total number of pixels a₁₁ at time point t₁₁ satisfies a₁₁>a₉. Therefore, a₁₁>aₘₐₓ, so the processor determines the total number of pixels a₁₁ at the current time point t₁₁ as the peak value b₁₁ at the current time point t₁₁. Therefore, the peak value b₁₁ is determined to satisfy b₁₁=a₁₁ (>b₁₀). After the peak value b₁₁ is determined, the process proceeds to step ST43.

In step ST43, the processor calculates the change amount c₁₁ of the peak value at time point t₁₁. Specifically, the processor sets the time window W including two pieces of data (i.e., a time window including the peak value b₁₁ at the current time point t₁₁ and the peak value b₁₀ at the time point t₁₀ immediately preceding the current time point t₁₁), and calculates the change amount c₁₁ between the two pieces of data b₁₀ and b₁₁ included in the time window W. The change amount c₁₁ can be expressed by the following formula.

$${c}_{11} = ( {b}_{11} - {b}_{10} ) / ( {t}_{11} - {t}_{10} )$$

After calculating the change amount c₁₁, the process proceeds to step ST44.

In step ST44, the index value d₁₁ at the current time point t₁₁ is calculated. The index value d₁₁ at the current time point t₁₁ can be calculated by d₁₁=c₁₁/b₁₁. After calculating the index value d₁₁, the process proceeds to step ST45.

In step ST45, the processor determines whether or not the current time point t₁₁ is the initial arrival time of the contrast agent on the basis of the index value d₁₁ at the current time point t₁₁ and the maximum value dₘₐₓ of the past index values. Specifically, the processor compares the index value d₁₁ at the current time point t₁₁ with the maximum value dₘₐₓ of the past index values. Then, if the index value d₁₁ at the current time point t₁₁ exceeds the maximum value dₘₐₓ of the past index values (d₁₁>dₘₐₓ), the processor determines that the current time point t₁₁ is the initial arrival time of the contrast agent, proceeds to step ST46, and updates the initial arrival time TA of the contrast agent from time point t₇ to time point t₁₁. On the other hand, if the index value d₁₁ at the current time point t₁₁ does not exceed the maximum value dₘₐₓ of past index values (d₁₁≤dₘₐₓ), the processor determines that the current time point t₁₁ is not the initial arrival time of the contrast agent, and proceeds to step ST50 without updating the initial arrival time TA of the contrast agent.

Here, the maximum index value dₘₐₓ obtained in the past satisfies dₘₐₓ=d₇, and the index value d₁₁ at time point t₁₁ satisfies d₁₁<d₇. Therefore, d₁₁≤dₘₐₓ, so the processor determines not to update the initial arrival time TA of the contrast agent, but to maintain the previously determined initial arrival time TA (=t₇), and proceeds to step ST50.

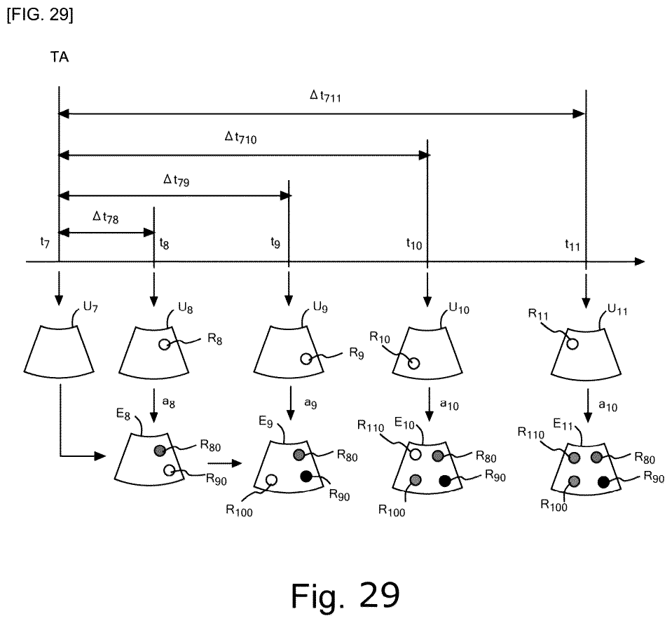

In step ST50, the processor creates a parametric image at time point t₁₁. FIG. 29 is an explanatory diagram of a method for creating a parametric image E₁₁ at time point t₁₁. Note that in FIG. 29, for convenience of explanation, it is assumed that at time point t₁₁, pixels exceeding the threshold value TH (total number of pixels an) appear to be concentrated in a region R11 of an ultrasound image U₁₁. Therefore, the processor identifies a region R110 corresponding to region R11 in the parametric image E₁₀, and assigns a color to the pixels of region R110 corresponding to the time difference Δt₇₁₁ between time point t₇ (initial arrival time TA of the contrast agent) and the current time point t₁₁, thereby creating a parametric image E₁₁ at the current time point t₁₁. Once the parametric image E₁₁ is displayed, the process proceeds to step ST60.

In step ST60, whether or not the examination has ended is determined. Herein, the examination has not yet ended, and thus the process proceeds to step ST48.

In step ST48, the processor increments i. Here, i=11, and thus i is incremented to 12. The process then returns to step ST30.

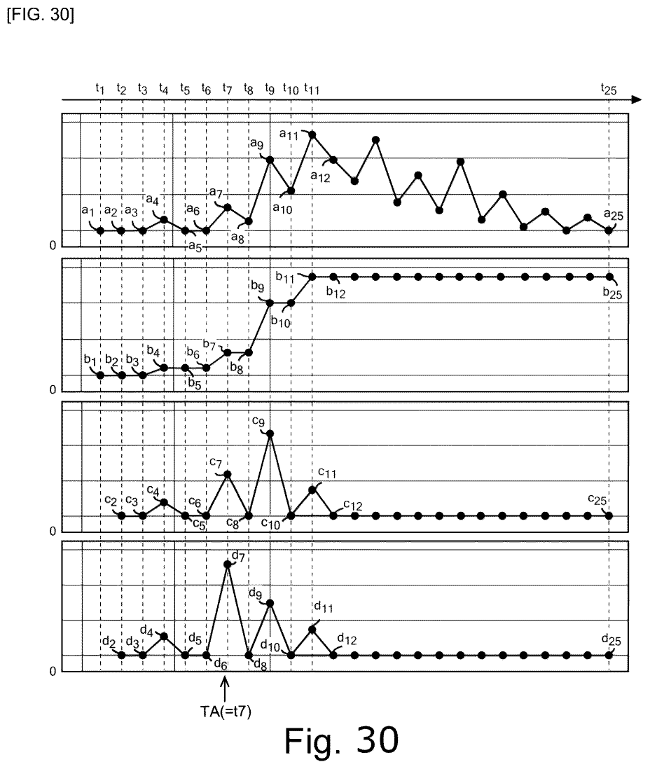

Similarly, the flow is repeatedly execute thereafter. FIG. 30 is a diagram depicting data obtained between time points t₁ and t₂₅. The total number of pixels aᵢ reaches a maximum value a₁₁ at time point t₁₁, but decreases gradually after time point t₁₁ without exceeding the maximum value a₁₁. Therefore, the peak value bᵢ is maintained at the same value from time point t₁₁ onward. Therefore, the index values d₁₂ to d₂₅ are zero, so the index values d₁₂ to d₂₅ do not exceed the maximum value d₇ of the index value, and thus the final updated value of the initial arrival time TA of the contrast agent is TA=t₇. Therefore, after time point t₇, the parametric image is repeatedly updated with the initial arrival time TA of the contrast agent being set to satisfy the reference time TA=t₇, and the contrast examination is completed.

In the present embodiment, in a contrast examination of a subject, the index value dᵢ is calculated each time an ultrasound image is acquired, and if the index value dᵢ at the current time point tᵢ exceeds the maximum value of the past index values, the initial arrival time TA of the contrast agent is updated. Therefore, the initial arrival time TA of the contrast agent can be automatically detected in real-time while a contrast examination is being performed on the subject. This eliminates the need for a user to analyze acquired ultrasound images to determine the initial arrival time of the contrast agent, thereby reducing the workload on the user during a contrast examination. Furthermore, the initial arrival time of the contrast agent can be detected during a contrast examination, so a parametric image can be created in parallel with the contrast examination. Therefore, the time required for analyzing an ultrasound image can be shortened.

Note that in the present embodiment, an index value dᵢ at a time point tᵢ is calculated, and if the index value dᵢ at the current time point tᵢ exceeds a maximum value dₘₐₓ of the past index values, the initial arrival time of the contrast agent is updated. Therefore, as depicted in FIG. 28, if the index value at the current time point tᵢ exceeds the maximum value dₘₐₓ of the past index values between times t₁ to t₆, the initial arrival time of the contrast agent can be determined even between times t₁ to t₆. In the present embodiment, time point t₄ is determined as the initial arrival time of the contrast agent. However, the contrast agent has not yet reached the examination site during the time points t₁ to t₆, so the index value d₄ at the time point t₄ is a sufficiently small value. On the other hand, the contrast agent flows into the examination site at time point t₇, so the index value d₇ at time point t₇ is large. Therefore, the index value d₇ at time point t₇ exceeds the index value d₄ at time point t₄, so the initial arrival time TA of the contrast agent can be updated from time point t₄ to time point t₇. Therefore, the parametric images being continuously generated referencing time point t₄ can be avoided after the initial arrival time TA (=t₇) of the contrast agent has passed.

Furthermore, in the present embodiment, the total number of pixels exceeding the threshold value Th increases rapidly at time point t₉ after time point t₇. However, the index value d₉ at time point t₉ is influenced by the peak value b₉, and is smaller than the index value d₇ at time point t₇, so the risk that time point t₉ is determined to be the initial arrival time TA of the contrast agent can be avoided.

Furthermore, in the present embodiment, the peak values b₁₁ to b₂₅ maintain the same values after time point t₁₁, so the change amount c₁₂ to c₂₅ is zero. Therefore, the index values d₁₂ to d₂₅ are zero (or a sufficiently small value) after time point t₁₂, so the index values d₁₂ to d₂₅ after time point t₁₂ are prevented from exceeding the index value d₇ at time point t₇. Therefore, the possibility that time points t₁₁ to t₂₅ will be determined to be the initial arrival times of the contrast agent can be avoided.

Note that in the present embodiment, the initial arrival time TA of the contrast agent is automatically detected in real-time while a contrast examination is being performed on the subject. However, the initial arrival time TA of the contrast agent may be automatically detected in real-time after the contrast examination is completed.

### DESCRIPTION OF CODES

1. Ultrasound diagnostic device
2. Ultrasonic probe
2a. Vibrating element
3. Transmission beamformer
4. Transmitter
5. Receiver
6. Reception beamformer
7. Processor
8. Display unit
9. Memory
10. User interface

1. An ultrasound diagnostic device, comprising one or more processors configured to:
compare, with a threshold value, a pixel value of each pixel included in an ultrasound image acquired at a current time point and counting the total number of pixels having a pixel value greater than the threshold value;
determine the total number of pixels obtained for the ultrasound image of the current time point as a peak value if the total number of pixels obtained for the ultrasound image of the current time point is greater than the maximum value of the total number of pixels obtained in the past, and performing a peak hold for holding the peak value at a time point immediately preceding the current time point as the peak value at the current time point if the total number of pixels obtained for the ultrasound image of the current time point is at the maximum value or less of the total number of pixels obtained in past;
set a time window including the peak value of the current time point and a past peak value, and calculating a change amount at the current time point in the peak value based on a plurality of peak values included in the time window;
calculate an index value of a current time point representative of the change amount at the current time point with respect to the peak value of the current time point;
determine whether the current time point is an initial arrival time of a contrast agent based on the index value of the current time point and the maximum value of the index values obtained in the past; and
repeatedly execute the process of counting the total number of pixels, the process of executing the peak hold, the process of calculating the change amount, the process of calculating the index value, and the process of determining, and then updating the initial arrival time of the contrast agent every time the current time is deemed to be the initial arrival time of the contrast agent.

2. The ultrasound diagnostic device according to claim 1, wherein the one or more processors is configured to count the total number of pixels, the process of executing the peak hold, the process of calculating the change amount, the process of calculating the index value, and the process of determining, every time an ultrasound image is acquired.

3. The ultrasound diagnostic device according to claim 1, wherein the one or more processors is configured to detect an initial arrival time of a contrast agent on the basis of an ultrasound image acquired at a current time point.

4. The ultrasound diagnostic device according to claim 3, wherein determining whether the current time point is an initial arrival time of a contrast agent includes determining whether an index value of the current time point exceeds the maximum value of index values obtained in the past.

5. The ultrasound diagnostic device according to claim 4, wherein the one or more processors is configured to, if the initial arrival time of the contrast agent is deemed to be updated, set the ultrasound image acquired at the current time point as a reference image for a parametric image.

6. The ultrasound diagnostic device according to claim 5, wherein the one or more processors is configured to create a parametric image if the index value of the current time point does not exceed the maximum value of index values obtained in the past.

7. The ultrasound diagnostic device according to claim 6, wherein the one or more processors is configured to:
determine whether examination is completed; and
if examination is deemed to not have been completed, determine whether an ultrasound image has been acquired, and executing a process of detecting the initial arrival time of the contrast agent with respect to the acquired ultrasound image.

8. The ultrasound diagnostic device according to claim 6, wherein the one or more processors is configured to, after the reference image for the parametric image is set, update the parametric image every time the index value of the current time point is deemed to not exceed the maximum value of index values obtained in the past.

9. The ultrasound diagnostic device according to claim 1, wherein the threshold value is determined on the basis of the maximum value of pixel values that can be displayed by each pixel.

10. A non-transitory computer-readable storing medium in which an instruction is stored, wherein
the instruction, when executed by one or a plurality of processors, causes the one or plurality of processors to execute the following:
comparing, with a threshold value, a pixel value of each pixel included in an ultrasound image acquired at a current time point and counting the total number of pixels having a pixel value greater than the threshold value;
determining the total number of pixels obtained for the ultrasound image of the current time point as a peak value if the total number of pixels obtained for the ultrasound image of the current time point is greater than the maximum value of the total number of pixels obtained in the past, and performing a peak hold for holding the peak value at a time point immediately preceding the current time point as the peak value at the current time point if the total number of pixels obtained for the ultrasound image of the current time point is at the maximum value or less of the total number of pixels obtained in past;
setting a time window including the peak value of the current time point and a past peak value, and calculating a change amount at the current time point in the peak value on the basis of a plurality of peak values included in the time window;
calculating an index value of a current time point representative of the change amount at the current time point with respect to the peak value of the current time point;
determining whether the current time point is an initial arrival time of a contrast agent on the basis of the index value of the current time point and a maximum value of the index values obtained in the past; and
repeatedly executing the step of counting the total number of pixels, the step of executing the peak hold, the step of calculating the change amount, the step of calculating the index value, and the step of determining, and then updating the initial arrival time of the contrast agent every time the current time is deemed to be the initial arrival time of the contrast agent.
